(19) United States
(12) Patent Application Publication
(10) Pub. No.: US 2024/0383921 A1
(43) Pub. Date: Nov. 21, 2024

(54) AMIDE AND UREA-CONTAINING TRICYCLIC KRAS INHIBITORS

(71) Applicant: Erasca, Inc., San Diego, CA (US)

(72) Inventors: Marcos Gonzalez-Lopez, San Diego, CA (US); Jean-Michel Vernier, San Diego, CA (US); Jun Feng, San Diego, CA (US); Benjamin Jones, San Diego, CA (US); Nicholas A. Isley, San Diego, CA (US); Ping Chen, San Diego, CA (US)

(21) Appl. No.: 18/571,138

(22) PCT Filed: Jun. 15, 2022
(86) PCT No.: PCT/US2022/033533

§ 371 Date: Dec. 15, 2023

Related U.S. Application Data
(60) Provisional application No. 63/211,493, filed on Jun. 16, 2021.

(51) Int. Cl. C07D 513/04; A61K 31/542; A61K 31/554; A61K 45/06

(52) U.S. Cl. CPC C07D 513/04; A61K 31/542; A61K 31/554; A61K 45/06

(57) ABSTRACT

The present embodiments provide compounds of Formula I, pharmaceutical compositions of the compounds, and methods for treating diseases such as cancer.

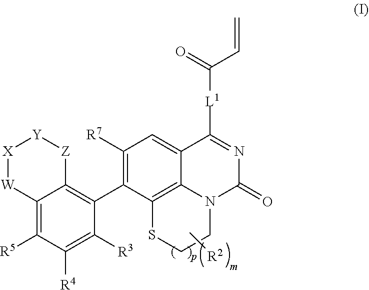

### REFERENCE TO SEQUENCE LISTING

This application incorporates by reference a Computer Readable Form (CRF) of a Sequence Listing in ASCII text format submitted with this application, entitled 055745-534001WO_SL_ST25.TXT, was created on Jun. 14, 2022, and is 3,656 bytes in size.

### BACKGROUND

Embodiments herein relate to compounds, compositions and methods for the treatment of RAS-mediated disease. In particular, embodiments herein relate to compounds and methods for treating diseases such as cancer via targeting oncogenic mutants of the K-RAS isoform.

Ras proteins are small guanine nucleotide-binding proteins that act as molecular switches by cycling between active GTP-bound and inactive GDP-bound conformations. Ras signaling is regulated through a balance between activation by guanine nucleotide exchange factors (GEFs), most commonly son of sevenless (SOS), and inactivation by GTPase-activating proteins (GAPs) such as neurofibromin or p120GAP. The Ras proteins play an important role in the regulation of cell proliferation, differentiation, and survival. Dysregulation of the Ras signaling pathway is almost invariably associated with disease. Hyper-activating somatic mutations in Ras are among the most common lesions found in human cancer. Most of these mutations have been shown to decrease the sensitivity of Ras to GAP stimulation and decrease its intrinsic GTPase activity, leading to an increase in the active GTP-bound population. Although mutation of any one of the three Ras isoforms (K-Ras, N-Ras, or H-Ras) has been shown to lead to oncogenic transformation, K-Ras mutations are by far the most common in human cancer. For example, K-Ras mutations are known to be often associated with pancreatic, colorectal and non-small-cell lung carcinomas. Similarly, H-Ras mutations are common in cancers such as papillary thyroid cancer, lung cancers and skin cancers. Finally, N-Ras mutations occur frequently in hepatocellular carcinoma.

K-Ras is the most frequently mutated oncoprotein in human cancers. Accordingly, there is a need to develop selective inhibitors of KRAS mutants. The present embodiments meet this and other needs.

### SUMMARY

In one aspect, the present embodiments provide compounds, or a pharmaceutically acceptable salt thereof, of Formula (Ia):

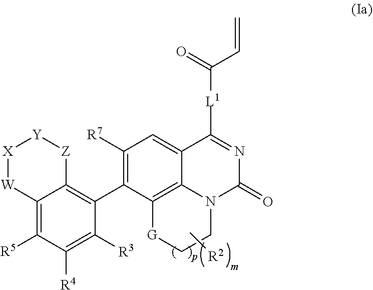

wherein
   wherein W, X, Y, and Z are independently selected from N, NH, CH, C═S, C═O, S or null; wherein null can only occur once, and at least one of W, X, Y, and Z is NH and at least one of W, X, Y, and Z adjacent to the NH is C═O;
   G is O or S;
   m is 1 or 2;
   p is 1 or 2;
   L¹ is

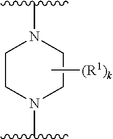

wherein k is an integer from 0 to 4; and each R¹ is independently selected from methyl, and cyanomethyl, C₂-C₄ alkyl, cyano, cycloalkyl, halo, haloalkyl, trifluoromethyl, and alkoxy; or any two R¹ combine to form a fused ring, bridge or spirocycle structure optionally comprising a heteroatom in the bridge or spirocycle selected from S, SO₂, O or N, and wherein the bridge or spirocycle structure is optionally substituted with oxo;
   each R² is independently selected from the group consisting of alkyl, N-alkylamino, N, N-dialkylamino, alkylamidoalkyl, arylamidoalkyl, —OCH₂CONRR′, heteroarylalkoxyalkyl, and heteroaryloxy, wherein R and R′ are independently selected from hydrogen, alkyl, and cycloalkyl, alkylsulfonamidoalkyl, arylsulfonamidoalkyl, N-alkyl aminoalkyl, N,N-dialkyl aminoalkyl, alkoxy, alkoxyalkyl, cycloalkyl, alkylcycloalkyl, hydroxyalkyl, halogen, haloalkyl, aryl, aryloxy, aralkyl, heteroaryl, heteroarylalkyl, heterocyclyl, heterocyclylalkyl, and heteroaryloxy any of which are optionally substituted; or when m is 2, two R² combine to form a spirocyclic 3-6-membered ring optionally containing 1 to 3 heteroatoms selected from N, O, or S;
   R³, R⁴, and R⁵, are independently selected from halogen, hydrogen, hydroxyl, alkoxy, alkyl, cycloalkyl, amino, N-alkylamino;
   R⁶ is H or methyl; and
R⁷ is alkyl, cyano, cycloalkyl, halogen, haloalkyl, trifluoromethyl, and alkoxy.

In another aspect, the present embodiments provide a pharmaceutical composition comprising a pharmaceutically effective amount of the compounds disclosed herein, or a pharmaceutically acceptable salt thereof, and a pharmaceutically acceptable excipient.

In another embodiment, the present embodiments provide a method of treating a subject having cancer, the cancer characterized by the presence of a KRAS G12C mutation, the method comprising administering to the subject a therapeutically effective amount of a compound disclosed herein, or a pharmaceutically acceptable salt thereof, or a pharmaceutical composition as disclosed herein.

In another embodiment, the present embodiments provide a method for manufacturing a medicament for treating a subject having cancer, the cancer characterized by the presence of a KRAS G12C mutation, the medicament comprising a compound disclosed herein, or a pharmaceutically acceptable salt thereof, or a a pharmaceutical composition as disclosed herein, is used.

In another embodiment, the present embodiments provide for the use of a compound disclosed herein, or a pharmaceutically acceptable salt thereof, or a a pharmaceutical composition as disclosed herein, for the manufacture of a medicament for the treatment of cancer in a subject, the cancer characterized by the presence of a KRAS G12C mutation.

In another embodiment, the present embodiments provide the compounds disclosed herein, or a pharmaceutically acceptable salt thereof, or a a pharmaceutical composition as disclosed herein, for use in the treatment of cancer in a subject, the cancer characterized by a KRAS G12C mutation.

### DETAILED DESCRIPTION

### I. General

The present embodiments provide inhibitors of KRAS G12C exhibiting good selectivity over wild-type KRAS and are useful for treating a cancer characterized by a KRAS G12C mutation.

### II. Definitions

Unless specifically indicated otherwise, all technical and scientific terms used herein have the same meaning as commonly understood by those of ordinary skill in the art to which the embodiments belong. In addition, any method or material similar or equivalent to a method or material described herein can be used in the practice of the present embodiments. For purposes of the present embodiments, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene. Alkenylen groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, isopropynylene, butynylene, sec-butynylene, pentynylene and hexynylene. Alkynylene groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The alkoxy component is as defined above. Examples of the alkoxyalkyl group include, but are not limited to, 2-ethoxy-ethyl and methoxymethyl.

“Alkylhydroxy” or “hydroxyalkyl” refers to an alkyl group, as defined above, where at least one of the hydrogen atoms is replaced with a hydroxy group. As for the alkyl group, alkylhydroxy groups can have any suitable number of carbon atoms, such as C₁₋₆. Exemplary alkylhydroxy groups include, but are not limited to, hydroxy-methyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Halogen” or “halo” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbomene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Cycloalkylene” refers to a cycloalkyl group having the number of carbon atoms indicated, and linking at least two other groups, i.e., a divalent radical. The two moieties linked to the cycloalkylene can be linked to the same atom or different atoms of the cycloalkylene group. Examples of cycloalkylene rings include cyclopropylene, cyclobutylene, cyclopentylene and cyclohexylene, among others. Cycloalkylene groups can be linked 1,1, 1,2, 1,3, or 1,4. The cyclohexylene ring, for example, can adopt a number of conformations, including the boat and chair conformations. The chair conformation of cyclohexylene can have substituents in an axial or equatorial orientation. The divalent nature of the cycloalkylenes results in cis and trans formations where cis refers to both substituents being on the same side (top or bottom) of the cycloalkylene ring, and where trans refers to the substituents being on on opposite sides of the cycloalkylene ring. For example, cis-1,2- and cis-1,4-cyclohexylene can have one substituent in the axial orientation and the other substituent in the equatorial orientation, while trans-1,2- and trans-1,4-cyclohexylene have both substituents in the axial or equatorial orientation. cis-1,3-cyclohexylene have both substituents in the axial or equatorial orientation, and trans-1,3-cyclohexylene can have one substituent in the axial orientation and the other substituent in the equatorial orientation. Cycloalkylene groups can be substituted or unsubstituted.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycloalkyl” or “heterocyclyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, dithiane, and hexahydro-1H-pyrrolizine. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, morpholine, and hexahydro-TH-pyrrolizine.

“Heterocyclalkylene” refers to a heterocyclalkyl group, as defined above, linking at least two other groups. The two moieties linked to the heterocyclalkylene can be linked to the same atom or different atoms of the heterocyclalkylene. Heterocycloalkylene groups can be substituted or unsubstituted.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above. Alkyl-heterocycloalkyl groups can be substituted or unsubstituted.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

“Arylene” refers to an aryl group, as defined above, linking at least two other groups. The two moieties linked to the aryl can be linked to the same atom or different atoms of the aryl. Arylene groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 5 to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Heteroarylene” refers to a heteroaryl group, as defined above, linking at least two other groups. The two moieties linked to the heteroaryl are linked to different atoms of the heteroaryl. Heteroarylene groups can be substituted or unsubstituted.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within. Alkyl-heteroaryl groups can be substituted or unsubstituted.

The groups defined above can optionally be substituted by any suitable number and type of substituents. Representative substituents include, but are not limited to, halogen, haloalkyl, haloalkoxy, —OR′, ═O, —OC(O)R′, —(O)R′, —O₂R′, —ONR′R″, —OC(O)NR′R″, ═NR′, ═N—OR′, —NR′R″, —NR″C(O)R′, —NR′—(O)NR″R″′, —NR″C(O)OR′, —NH—(NH₂)═NH, —NR′C(NH₂)═NH, —NH—(NH₂)═NR′, —SR′, —S(O)R′, —S(O)₂R′, —S(O)₂NR′R″, —NR′S(O)₂R″, —N ₃ and —NO₂. R′, R″ and R″′ each independently refer to hydrogen, unsubstituted alkyl, such as unsubstituted C₁₋₆ alkyl. Alternatively, R′ and R″, or R″ and R″′, when attached to the same nitrogen, are combined with the nitrogen to which they are attached to form a heterocycloalkyl or heteroaryl ring, as defined above.

“Salt” refers to acid or base salts of the compounds, which can be used in the methods disclosed herein. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds disclosed herein are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present embodiments.

Certain compounds disclosed herein possess asymmetric carbon atoms (optical centers) or double bonds; the racemates, diastereomers, geometric isomers and individual isomers are all intended to be encompassed within the scope of the present embodiments.

“Hydrate” refers to a compound that is complexed to at least one water molecule. The compounds disclosed herein can be complexed with from 1 to 10 water molecules.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

“Pharmaceutically acceptable excipient” refers to a substance that aids the administration of an active agent to and absorption by a subject. Pharmaceutical excipients useful in the present embodiments include, but are not limited to, binders, fillers, disintegrants, lubricants, coatings, sweeteners, flavors and colors. One of skill in the art will recognize that other pharmaceutical excipients are useful in the present embodiments.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Subject” refers to animals such as mammals, including, but not limited to, primates (e.g., humans), cows, sheep, goats, horses, dogs, cats, rabbits, rats, mice and the like. In certain embodiments, the subject is a human.

### II. Compounds

The present embodiments provide compounds, and pharmaceutically acceptable salts thereof, of Formula (I):

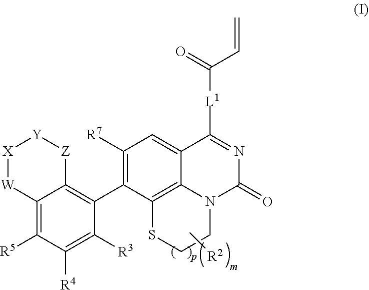

wherein
   wherein W, X, Y, and Z are independently selected from N, NH, CH, C═O, C═S, CNH₂, CNHMe, S or null; wherein null can only occur once, and at least one of W, X, Y, and Z is NH and at least one of W, X, Y, and Z adjacent to the NH is C═O;
   m is 1 or 2;
   p is 1 or 2;
   L¹ is

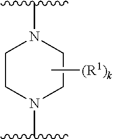

wherein k is an integer from 0 to 4; and each R¹ is independently selected from methyl, and cyanomethyl, C₂-C₄ alkyl, cyano, cycloalkyl, halo, haloalkyl, trifluoromethyl, and alkoxy; or any two R¹ combine to form a fused ring, bridge or spirocycle structure optionally comprising a heteroatom in the bridge or spirocycle selected from S, SO₂, O or N, and wherein the bridge or spirocycle structure is optionally substituted with oxo;
   each R² is independently selected from the group consisting of alkyl, N-alkylamino, N, N-dialkylamino, alkylamidoalkyl, arylamidoalkyl, —OCH₂CONRR′, heteroarylalkoxyalkyl, and heteroaryloxy, wherein R and R′ are independently selected from hydrogen, alkyl, and cycloalkyl, alkylsulfonamidoalkyl, arylsulfonamidoalkyl, N-alkyl aminoalkyl, N,N-dialkyl aminoalkyl, alkoxy, alkoxyalkyl, cycloalkyl, alkylcycloalkyl, hydroxyalkyl, halogen, haloalkyl, aryl, aryloxy, aralkyl, heteroaryl, heteroarylalkyl, heterocyclyl, heterocyclylalkyl, and heteroaryloxy any of which are optionally substituted; or when m is 2, two R² combine to form a spirocyclic 3-6-membered ring optionally containing 1 to 3 heteroatoms selected from N, O, or S;
   R³, R⁴, and R⁵, are independently selected from halogen, hydrogen, hydroxyl, alkoxy, alkyl, cycloalkyl, amino, N-alkylamino;
   R⁶ is H or methyl; and
   R⁷ is alkyl, cyano, cycloalkyl, halogen, haloalkyl, trifluoromethyl, and alkoxy.

In embodiments, n is 1. In embodiments, n is 2. In embodiments, n is 3. In embodiments, n is 4.

In embodiments, p is 1. In embodiments, p is 2.

In embodiments, L¹ selected from:

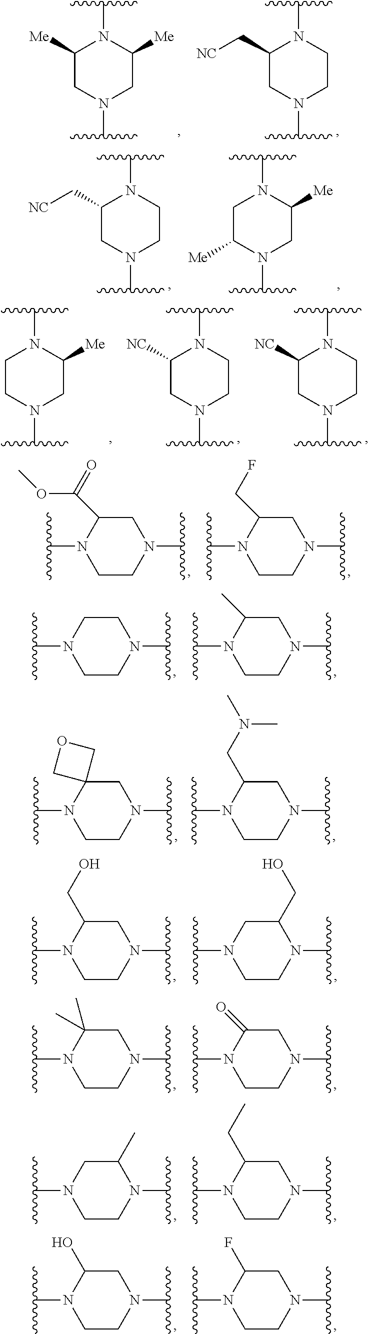

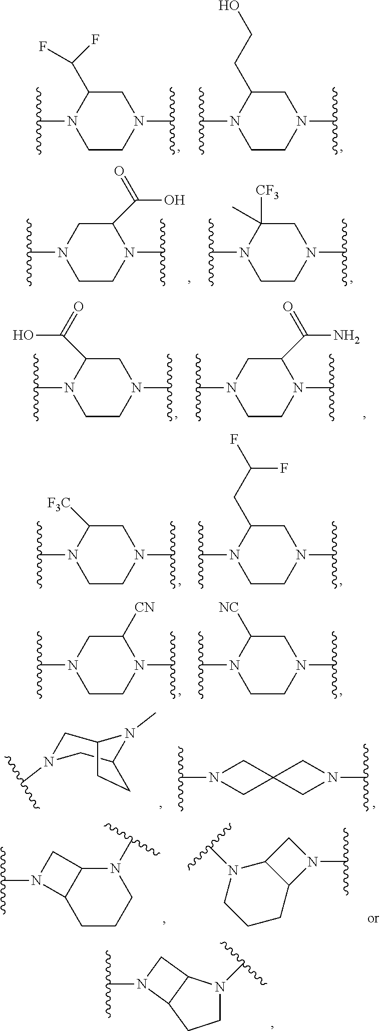

wherein

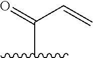 [SMILES: *C(=O)C=C]

is attached through either of the two nitrogen atoms of L¹.

In embodiments, R² is selected from methoxy, amino, MeOCH₂—, EtOCH₂-, MeO(CH₂)₂NH—,

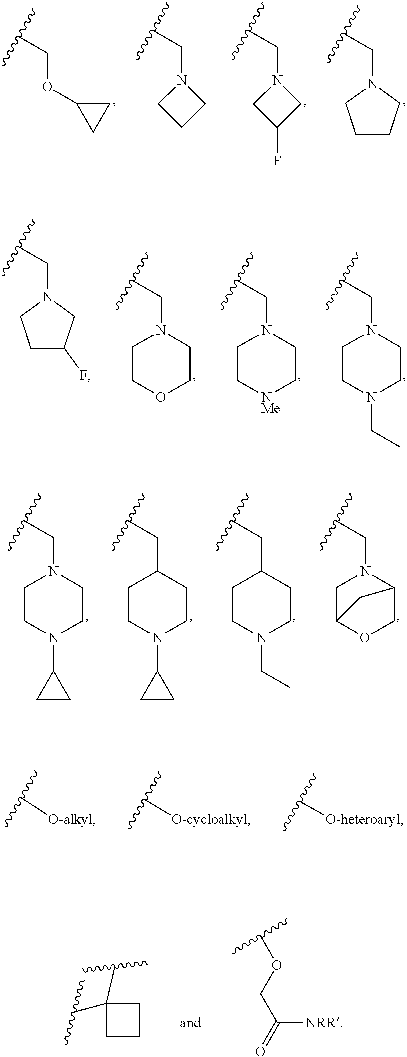

In embodiments, W, X, Y and Z define the heterocycle:

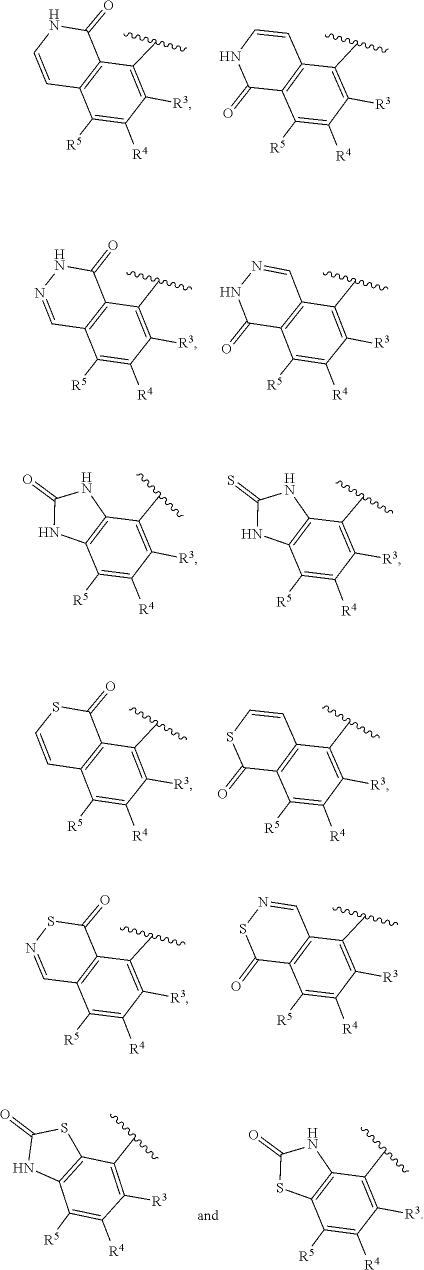

In embodiments, X and Y define the heterocycle:

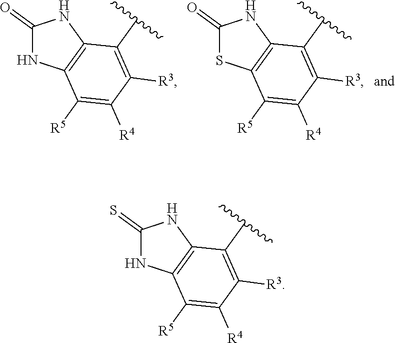

In embodiments, compounds disclosed herein include the following compounds or pharmaceutically acceptable salt thereof:

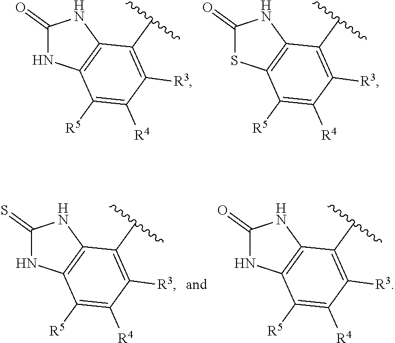

In embodiments, compounds disclosed herein include the following further compounds, or pharmaceutically acceptable salt thereof:

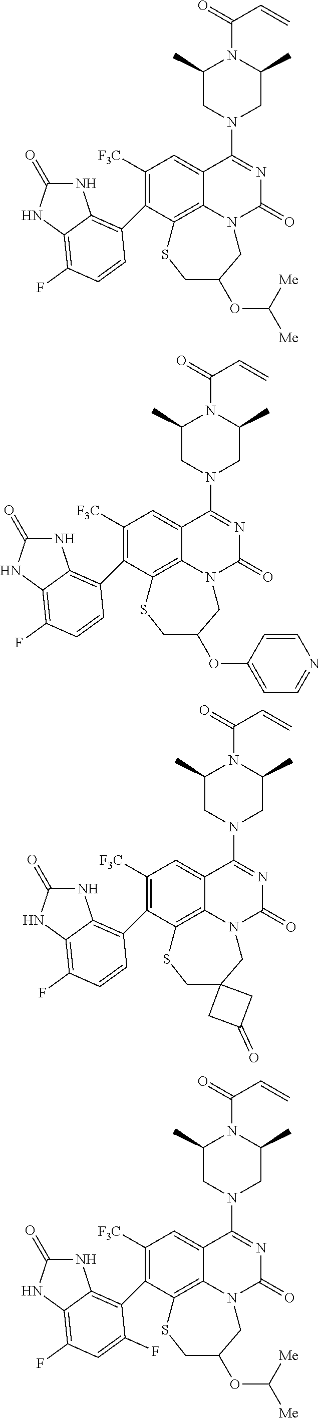

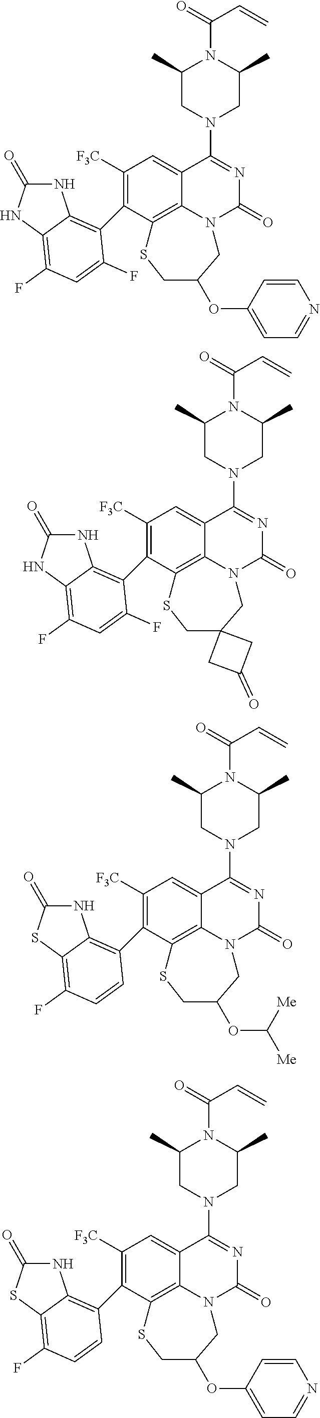

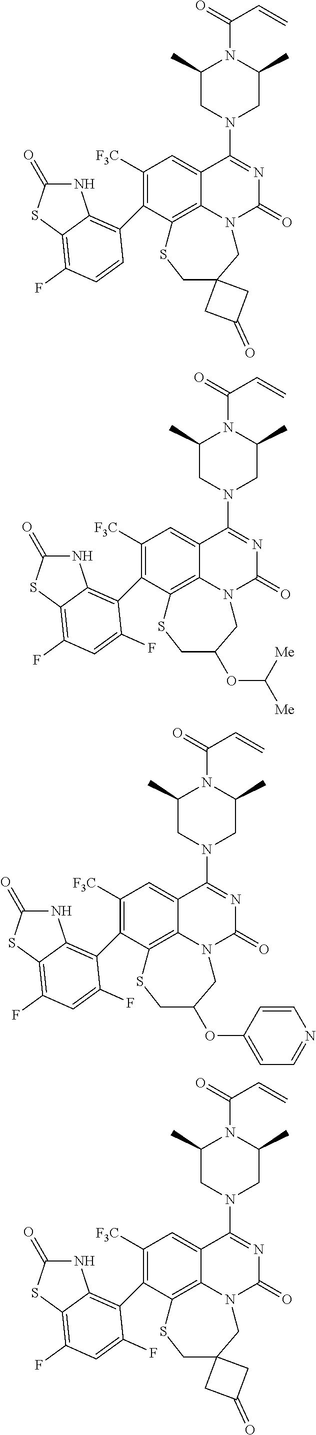

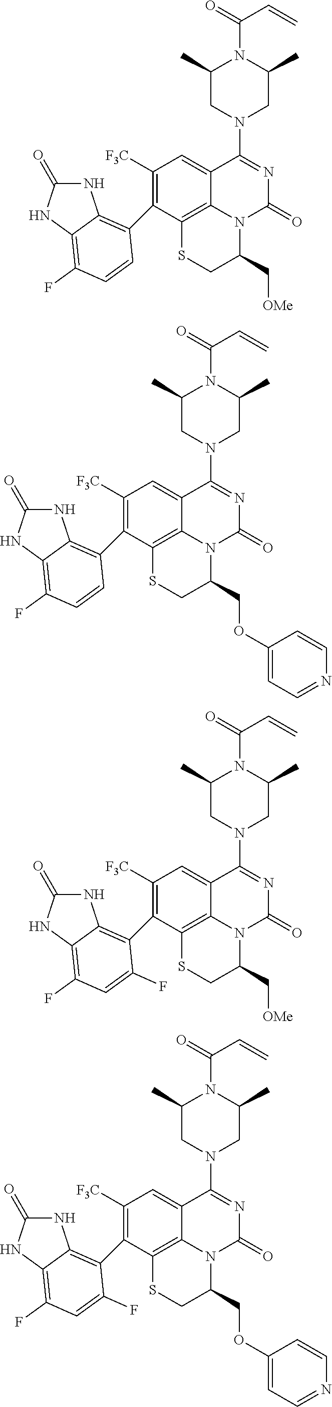

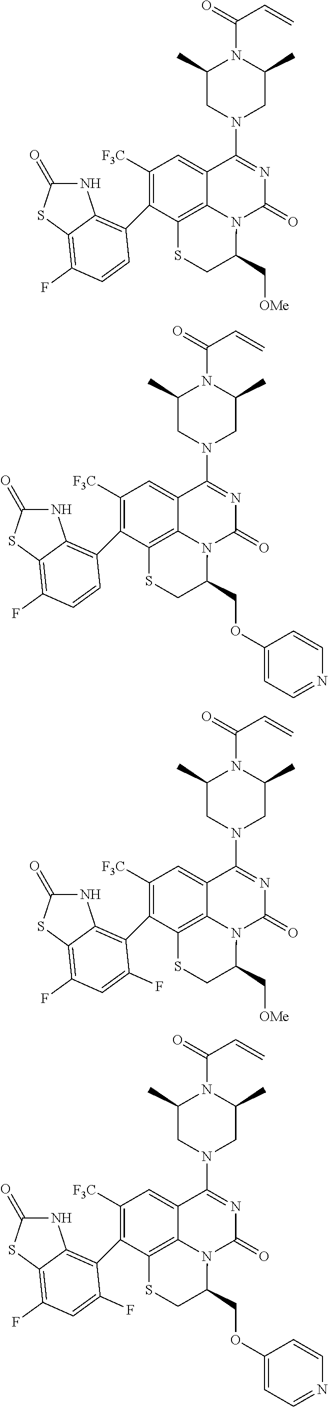

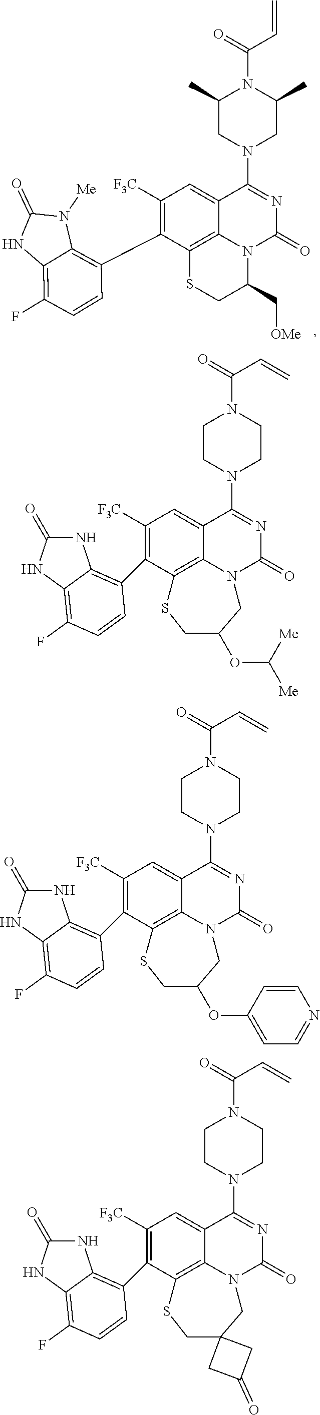

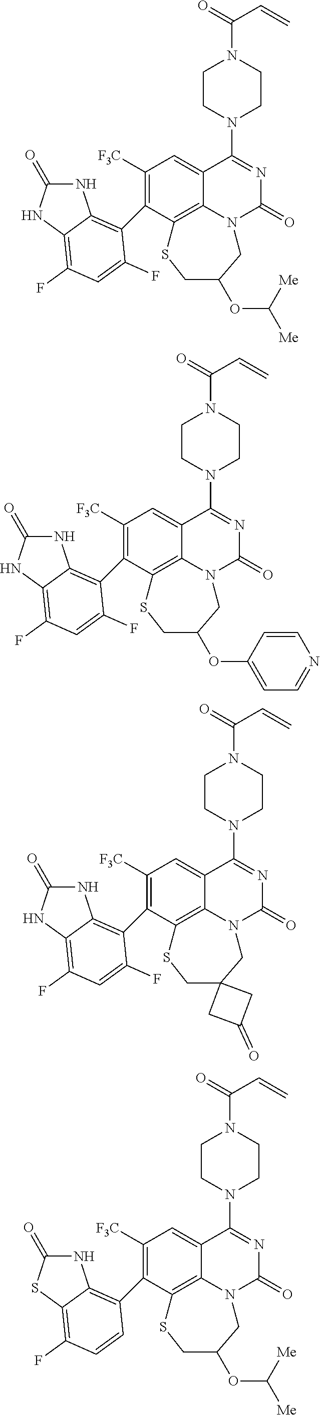

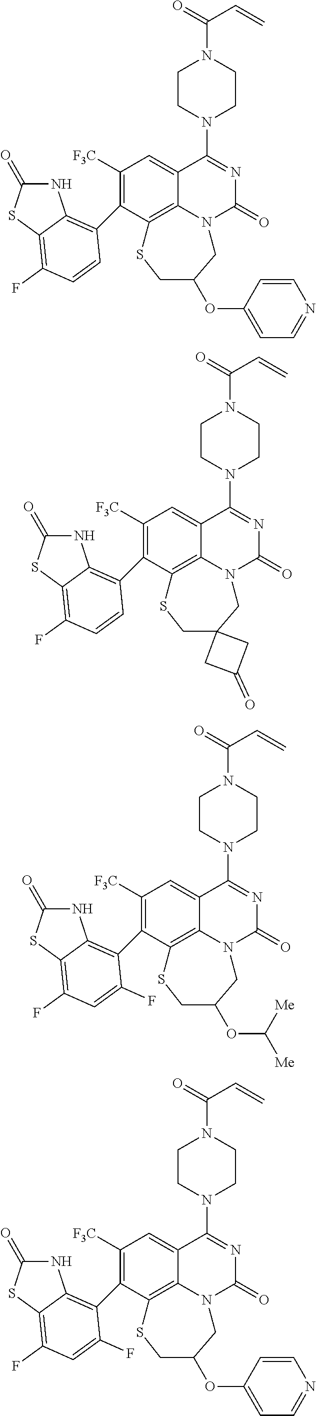

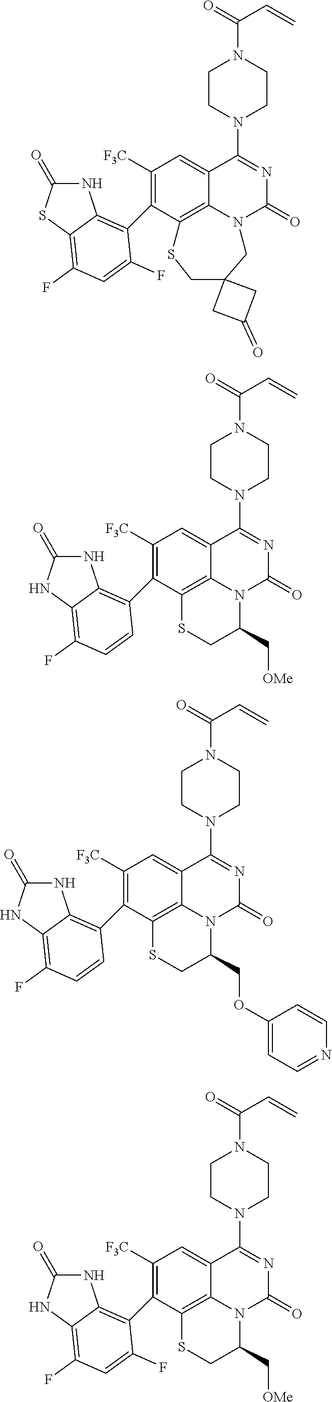

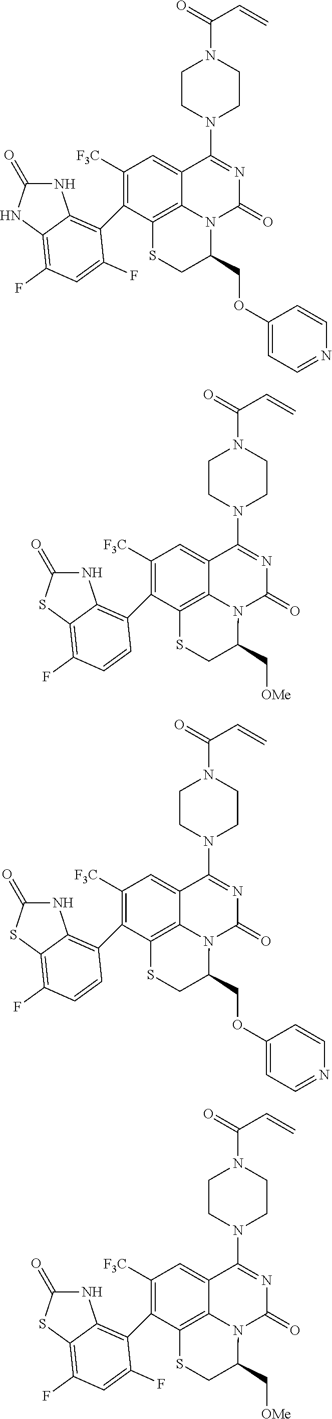

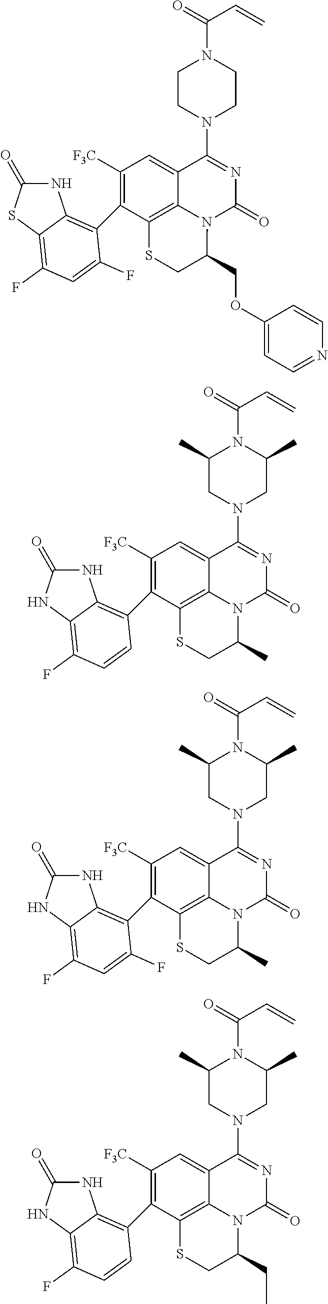

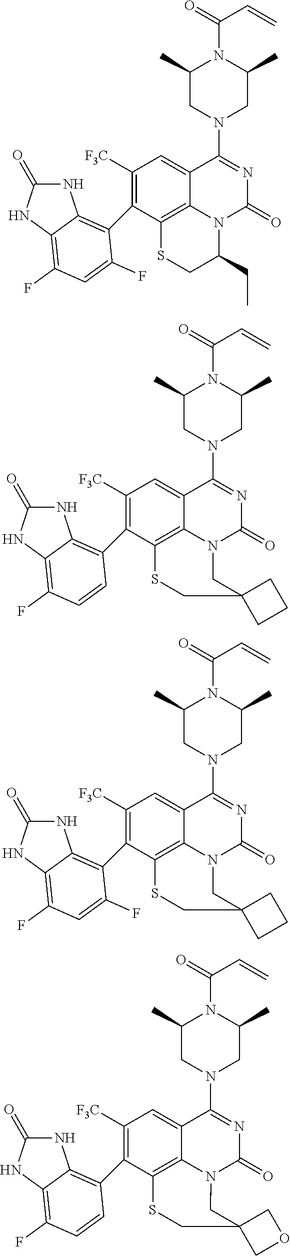

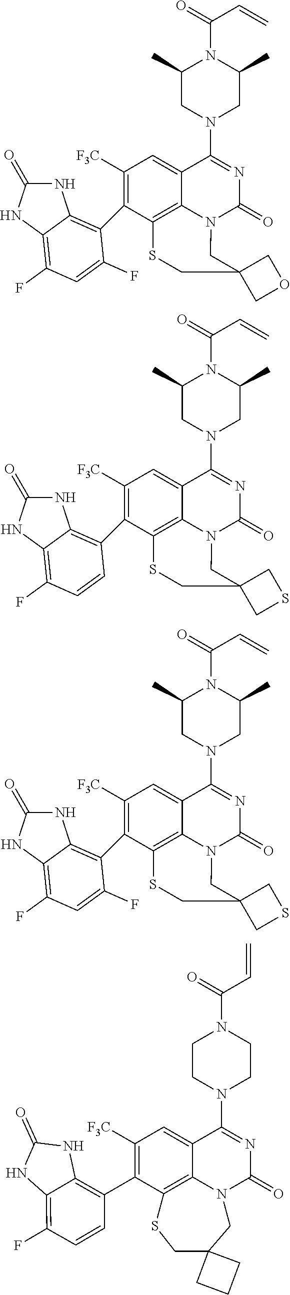

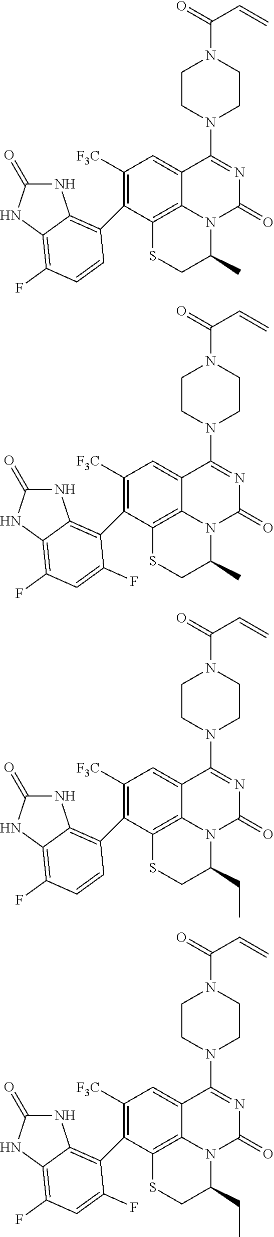

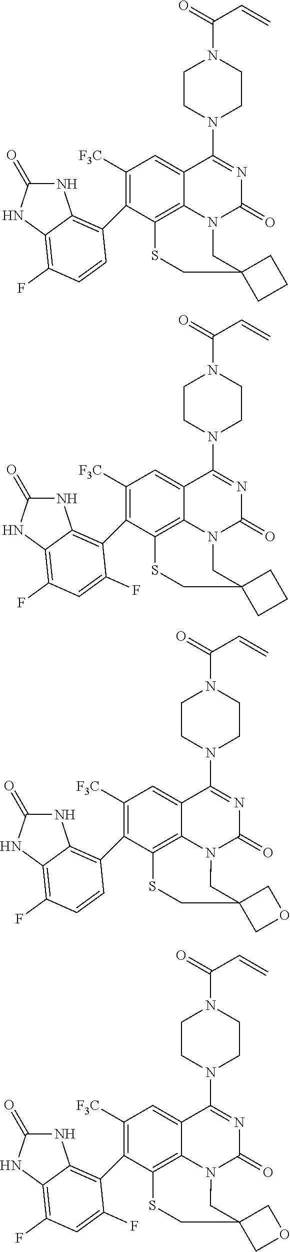

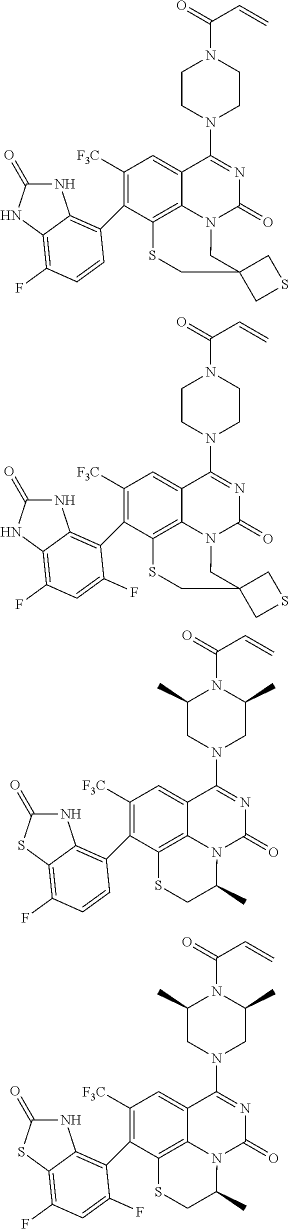

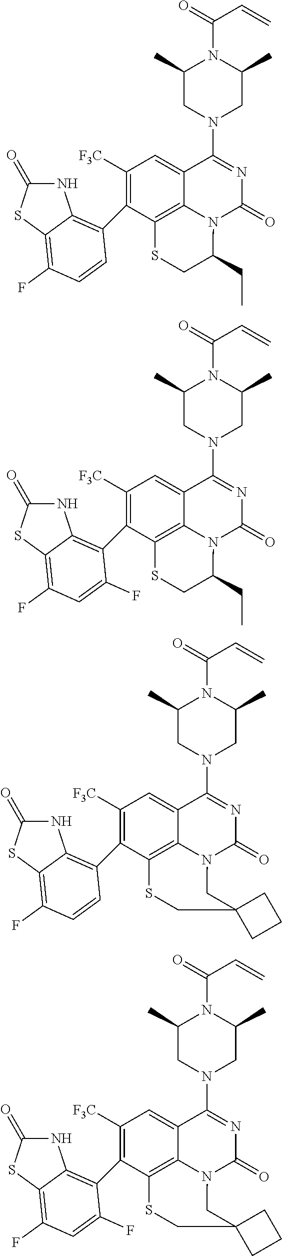

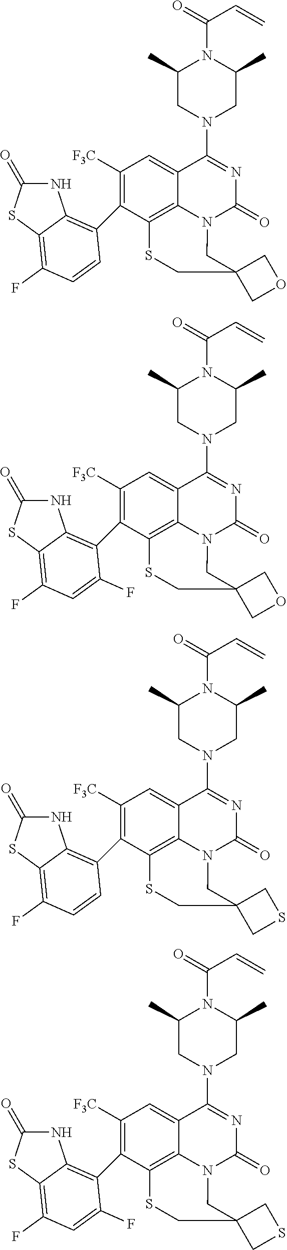

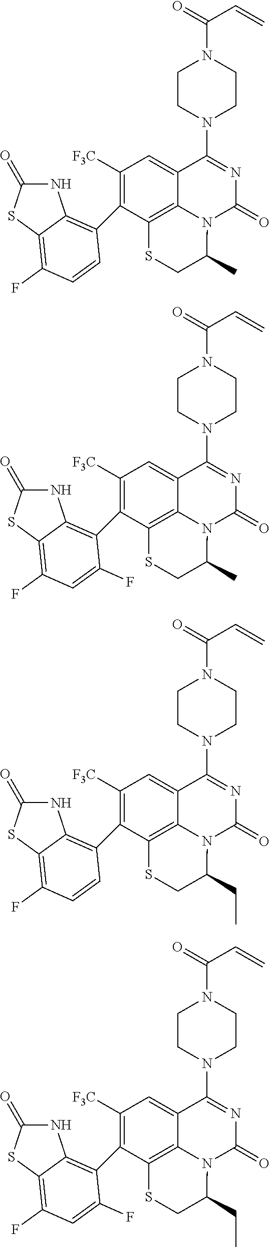

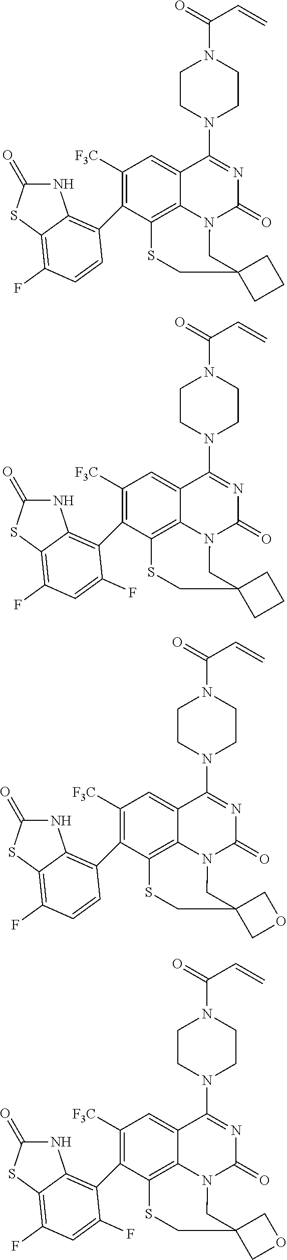

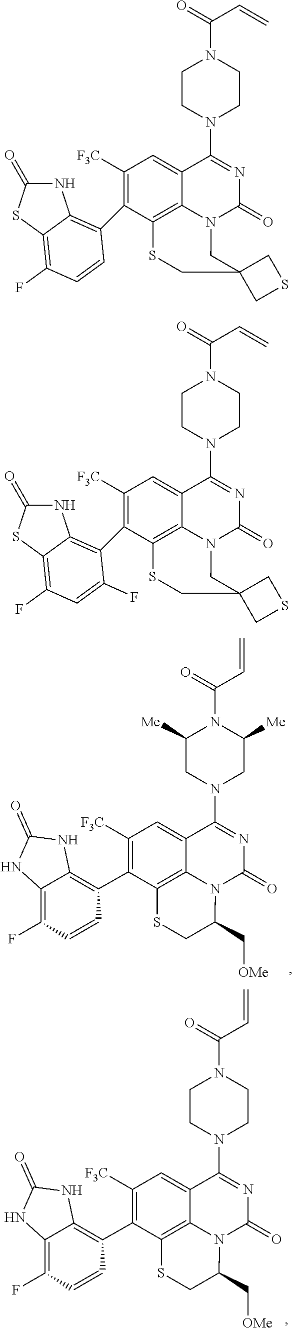

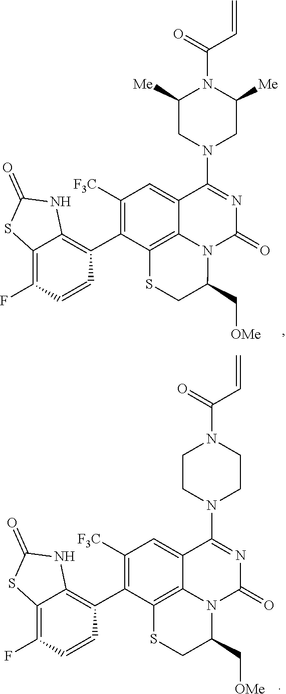

The compounds disclosed herein can exist as salts. The present embodiments include such salts, which can be pharmaceutically acceptable salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds disclosed herein contain both basic and acidic functionalities that allow the compounds to be converted into either base or acid addition salts.

Other salts include acid or base salts of the compounds used in the methods of the present embodiments. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts includes salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds disclosed herein contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds disclosed herein can exist in unsolvated forms as well as solvated forms, including hydrated forms. In general, the solvated forms are equivalent to unsolvated forms and are encompassed within the scope of the present embodiments. Certain compounds disclosed herein may exist in multiple crystalline or amorphous forms. In general, all physical forms are equivalent for the uses contemplated by the present embodiments and are intended to be within the scope of the present embodiments.

Certain compounds disclosed herein possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present embodiments. The compounds disclosed herein do not include those which are known in art to be too unstable to synthesize and/or isolate. The present embodiments are meant to include compounds in racemic and optically pure forms. Optically active (R)- and (S)-, or (D)- and (L)-isomers may be prepared using chiral synthons or chiral reagents, or resolved using conventional techniques. The compounds disclosed herein can be provided as a mixture of atropisomers or can be pure atropisomers.

Isomers include compounds having the same number and kind of atoms, and hence the same molecular weight, but differing in respect to the structural arrangement or configuration of the atoms.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the embodiments.

Unless otherwise stated, the compounds disclosed herein may also contain unnatural proportions of atomic isotopes at one or more of the atoms that constitute such compounds. For example, the compounds disclosed herein may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds disclosed herein, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds disclosed herein. Additionally, prodrugs can be converted to the compounds disclosed herein by chemical or biochemical methods in an ex vivo environment. For example, prodrugs can be slowly converted to the compounds disclosed herein when placed in a transdermal patch reservoir with a suitable enzyme or chemical reagent.

Compounds disclosed herein can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds.) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40. The following synthetic reaction schemes are merely illustrative of some methods by which the compounds disclosed herein can be synthesized, and various modifications to these synthetic reaction schemes can be made and will be suggested to one skilled in the art having referred to the disclosure contained herein.

For illustrative purposes, reaction Schemes below provide routes for synthesizing the compounds disclosed herein as well as key intermediates. For a more detailed description of the individual reaction steps, see the Examples section below. Those skilled in the art will appreciate that other synthetic routes may be used. Although some specific starting materials and reagents are depicted in the Schemes and discussed below, other starting materials and reagents can be substituted to provide a variety of derivatives or reaction conditions. In addition, many of the compounds prepared by the methods described below can be further modified in light of this disclosure using conventional chemistry well known to those skilled in the art.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, or, about 20° C.

Some compounds in following schemes are depicted with generalized substituents; however, one skilled in the art will immediately appreciate that the nature of the substituents can varied to afford the various compounds contemplated in the present embodiments. Moreover, the reaction conditions are exemplary and alternative conditions are well known. The reaction sequences in the following examples are not meant to limit the scope of the embodiments as set forth in the claims.

### IV. Pharmaceutical Formulations

In some embodiments, pharmaceutical compositions comprise a compound of any one of the compounds disclosed herein and a pharmaceutically acceptable excipient.

In some embodiments, there is provided a pharmaceutical composition comprising a pharmaceutically effective amount of a compound of Formula (I), or a pharmaceutically acceptable salt thereof, and a pharmaceutically acceptable excipient.

In some embodiments, the pharmaceutical composition further comprises an additional therapeutic agent.

In some embodiments, the additional therapeutic agent is a chemotherapeutic agent. In some embodiments, the chemotherapeutic agent is an anti-microtubule agent, a platinum coordination complex, a alkylating agent, an antibiotic agent, a topoisomerase II inhibitor, a antimetabolite, a topoisomerase I inhibitor, a hormone or hormonal analogue, a signal transduction pathway inhibitor, a non-receptor tyrosine kinase angiogenesis inhibitor, a immunotherapeutic agent, a proapoptotic agent, an inhibitor of LDH-A, an inhibitor of fatty acid biosynthesis, a cell cycle signalling inhibitor, a HDAC inhibitor, a proteasome inhibitor, or an inhibitor of cancer metabolism. In some embodiments, the chemotherapeutic agent is cisplatin, carboplatin, doxorubicin, ionizing radiation, docetaxel or paclitaxel.

The compounds disclosed herein can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds disclosed herein can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds disclosed herein can be administered transdermally. The compounds disclosed herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present embodiments also provide pharmaceutical compositions including one or more pharmaceutically acceptable carriers and/or excipients and either a compound of Formula I, or a pharmaceutically acceptable salt of a compound of Formula I.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other exceipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations can also be used orally using, for example, push-fit capsules made of gelatin, as well as soft, sealed capsules made of gelatin and a coating such as glycerol or sorbitol. Push-fit capsules can contain the compounds disclosed herein mixed with a filler or binders such as lactose or starches, lubricants such as talc or magnesium stearate, and, optionally, stabilizers. In soft capsules, the compounds disclosed herein may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compounds disclosed herein in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds disclosed herein can be delivered by transdermally, by a topical route, formulated as applicator sticks, solutions, suspensions, emulsions, gels, creams, ointments, pastes, jellies, paints, powders, and aerosols.

The compounds disclosed herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical formulations of the compounds disclosed herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

The pharmaceutical formulations of the compounds disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

In some embodiments, the formulations of the compounds disclosed herein can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR and/or MR modulator and disease or condition treated.

Single or multiple administrations of the compounds disclosed herein formulations can be administered depending on the dosage and frequency as required and tolerated by the patient. The formulations should provide a sufficient quantity of active agent to effectively treat the disease state. Thus, in one embodiment, the pharmaceutical formulations for oral administration of the compounds disclosed herein is in a daily amount of between about 0.5 to about 30 mg per kilogram of body weight per day. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compounds disclosed herein for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987).

The compounds described herein can be used in combination with one another, with other active agents known to be useful in modulating a glucocorticoid receptor, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another.

After a pharmaceutical composition including a compound disclosed herein has been formulated in one or more acceptable carriers, it can be placed in an appropriate container and labeled for treatment of an indicated condition. For administration of the compounds of Formula I, such labeling would include, e.g., instructions concerning the amount, frequency and method of administration.

In some embodiments, the compositions disclosed herein are useful for parenteral administration, such as intravenous (IV) administration or administration into a body cavity or lumen of an organ. The formulations for administration will commonly comprise a solution of the compositions disclosed herein dissolved in one or more pharmaceutically acceptable carriers. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, tonicity adjusting agents, e.g., sodium acetate, sodium chloride, potassium chloride, calcium chloride, sodium lactate and the like. The concentration of the compositions in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions disclosed herein can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions disclosed herein into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### V. Methods

In some embodiments, there is provided a method of treating a disorder or condition in a subject, the method comprising administering to the human a therapeutically effective amount of a compound disclosed herein, or a pharmaceutically acceptable salt thereof, or a pharmaceutical composition as disclosed herein.

In some embodiments, there is provided a method for inhibiting KRAS G12C activity in a cell, comprising contacting the cell in which inhibition of KRAS G12C activity is desired with an effective amount of a compound disclosed herein or a pharmaceutically acceptable salt thereof.

In some embodiments, there is provided a method for inhibiting KRAS G12C activity in a cell, comprising contacting the cell in which inhibition of KRAS G12C activity is desired with the pharmaceutical composition disclosed herein.

In some embodiments, there is provided a method for treating a KRAS G12C-associated cancer comprising administering to a patient in need thereof a therapeutically effective amount of a compound disclosed herein, or a pharmaceutically acceptable salt thereof.

In some embodiments, there is provided a method for treating a KRAS G12C-associated cancer comprising administering to a patient in need thereof the pharmaceutical composition disclosed herein.

In some embodiments, there is provided a method of treating a subject having cancer, the cancer characterized by the presence of a KRAS G12C mutation, the method comprising administering to the human a therapeutically effective amount of a compound of any one of Formula (I) or Formula (II), or a pharmaceutically acceptable salt thereof, or a a pharmaceutical composition as disclosed herein.

In some embodiments, there is provided a method for manufacturing a medicament for treating a subject having cancer, the cancer characterized by the presence of a KRAS G12C mutation, the compound comprising Formula (I) or Formula (II), or a pharmaceutically acceptable salt thereof, or a pharmaceutical composition.

In some embodiments, there is provided a use of a compound of Formula (I) or Formula (II), or a pharmaceutically acceptable salt thereof, or a a pharmaceutical composition as disclosed herein, for the manufacture of a medicament for the treatment in a human having cancer, the cancer characterized by the presence of a KRAS G12C mutation.

In some embodiments, there are provided compounds of Formula (I) or Formula (II), or a pharmaceutically acceptable salt thereof, or a a pharmaceutical composition as disclosed herein, for use in the treatment of a subject having cancer, the cancer characterized by the presence of a KRAS G12C mutation.

In some embodiments, there is provided a method for treating cancer in a patient in need thereof, the method comprising (a) determining that the cancer is associated with a KRAS G12C mutation (e.g., a KRAS G12C-associated cancer); and (b) administering to the patient a therapeutically effective amount of a compound disclosed herein.

In some embodiments, there is provided a method for treating cancer in a patient in need thereof, the method comprising (a) determining that the cancer is associated with a KRas G12C mutation (e.g., a KRAS G12C-associated cancer); and (b) administering to the patient the pharmaceutical composition disclosed herein.

In some embodiments, the cancer is Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

In some embodiments, the cancer is non-small cell lung cancer, small cell lung cancer, colorectal cancer, rectal cancer, or pancreatic cancer.

In certain embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The compounds of Formula (I) or Formula (II), or a pharmaceutically acceptable salt thereof, can be inhibitors of KRAS G12C. For example, the inhibition constant (Ki) of the compounds disclosed herein can be less than about 50 μM, or less than about 40, 30, 20, 10, 9, 8, 7, 6, 5, 4, 3, 2, or less than about 1 μM. The inhibition constant (Ki) of the compounds disclosed herein can be less than about 1,000 nM, or less than about 900, 800, 700, 600, 500, 400, 300, 200, 100, 90, 80, 70, 60, 50, 40, 30, 20, 10, 9, 8, 7, 6, 5, 4, 3, 2, or less than about 1 nM. The inhibition constant (Ki) of the compounds disclosed herein can be less than about 1 nM, or less than about 0.9, 0.8, 0.7, 0.6, 0.5, 0.4, 0.3, 0.2, or less than about 0.1 nM.

The compounds of Formula (I) or Formula (II), or a pharmaceutically acceptable salt thereof, can be selective inhibitors of KRAS G12C. For example, KRAS G12C inhibition constant (IC50) of the compounds disclosed herein can be at least 2-fold less than the inhibition constant of one or more of KRAS wild-type, or NRAS, or HRAS, or at least 3, 4, 5, 6, 7, 8, 9, 10, 20, 30, 40, 50, 60, 70, 80, 90 or 100-fold less. The KRAS G12C inhibition constant (Ki) of the compounds disclosed herein can also be at least 100-fold less than the inhibition constant of one or more of KRAS wild-type, or NRAS, or HRAS, or at least 200, 300, 400, 500, 600, 700, 800, 900, 1000, or 10,000-fold less.

### A. Cancer Combination Therapies

The compounds disclosed herein or salts thereof may be employed alone or in combination with other agents for treatment. For example, the second agent of the pharmaceutical combination formulation or dosing regimen may have complementary activities to the compounds disclosed herein such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound disclosed herein or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Those additional agents may be administered separately from an inventive compound-containing composition, as part of a multiple dosage regimen. Alternatively, those agents may be part of a single dosage form, mixed together with a compound disclosed herein, in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with embodiments herein. For example, a compound disclosed herein may be administered with another therapeutic agent simultaneously or sequentially in separate unit dosage forms or together in a single unit dosage form. Accordingly, the present embodiments provide a single unit dosage form comprising a compound of Formula I, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. In certain embodiments, compositions disclosed herein are formulated such that a dosage of between 0.01-100 mg/kg body weight/day of an inventive can be administered.

Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

In one embodiment, the treatment method includes the co-administration of a compound disclosed herein or a pharmaceutically acceptable salt thereof and at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signalling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylomithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds disclosed herein include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁ λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA@) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C₁₁ (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Chemotherapeutic agents also include treatments for Alzheimer's Disease such as donepezil hydrochloride and rivastigmine; treatments for Parkinson's Disease such as L-DOPA/carbidopa, entacapone, ropinrole, pramipexole, bromocriptine, pergolide, trihexephendyl, and amantadine; agents for treating multiple sclerosis (MS) such as beta interferon (e.g., Avonex® and Rebif©), glatiramer acetate, and mitoxantrone; treatments for asthma such as albuterol and montelukast sodium; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

Additionally, chemotherapeutic agents include pharmaceutically acceptable salts, acids or derivatives of any of chemotherapeutic agents, described herein, as well as combinations of two or more of them.

### VI. EXAMPLES

### Synthetic Procedures

### General Procedure

The compounds of Formula I may be prepared from commercially available reagents using the synthetic methods and reaction schemes herein, or using other reagents and conventional methods well known to those skilled in the art. For instance, compounds of the present invention may be prepared according to the general reactions in Scheme I

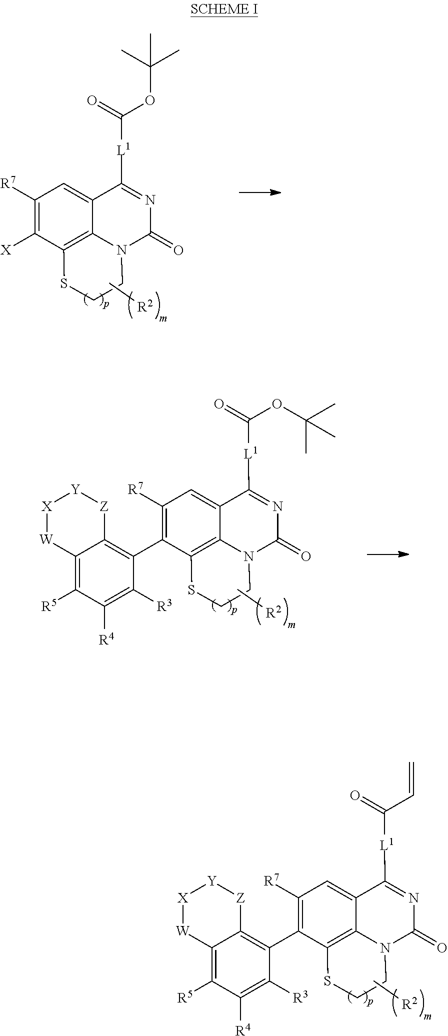

TABLE 1
Example Compounds

| | | | MS | |
|---|---|---|---|---|
| Ex. | Structure | IUPAC Name | (M + H)⁺ | ¹H-NMR |
| 1 | 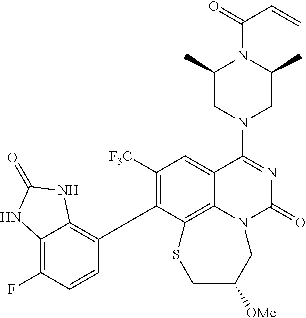 [SMILES: C=CC(=O)N1[C@H](C)CN(c2nc(=O)n3c4c(c(Cc5ccc(F)c6[nH]c(=O)[nH]c56)c(C(F)(F)F)cc24)SC[C@@H](OC)C3)C[C@@H]1C] | (3S)-8-((3S,5R)-4- acryloyl-3,5- dimethylpiperazin-1- yl)-11-(7-fluoro-2-oxo- 2,3-dihydro-1H- benzo[d]imidazol-4-yl)- 3-methoxy-10- (trifluoromethyl)-3,4- dihydro-2H,6H- [1,4]thiazepino[2,3,4- ij]quinazolin-6-one | 633.1 | ¹H NMR (400 MHz, DMSO- d₆) δ 11.35 (s, 1H), 10.85 (s, 1H), 8.03-7.99 (m, 1H), 6.98- 6.94 (m, 1H), 6.88-6.72 (m, 2H), 6.20 (dd, J = 16.0, 4.0 Hz, 1H), 5.75 (dd, J = 8.0, 4.0 Hz, 1H), 4.61-4.55 (m, 3H), 4.11-4.05 (m, 2H), 3.86-3.81 (m, 1H), 3.34-3.30 (m, 7H), 3.14 (m, 1H), 1.43-1.38 (m, 6H) |
| 2 | 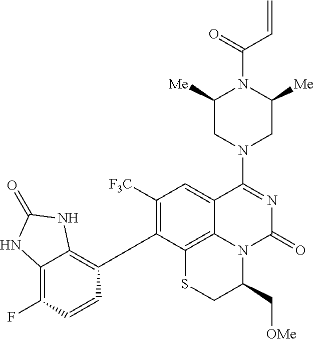 | (3S,10S)-7-((3S,5R)-4- acryloyl-3,5- dimethylpiperazin-1- yl)-10-(7-fluoro-2-oxo- 2,3-dihydro-1H- benzo[d]imidazol-4-yl)- 3-(methoxymethyl)-9- (trifluoromethyl)-2,3- dihydro-5H- [1,4]thiazino[2,3,4- ij]quinazolin-5-one | 633.2 | ¹H NMR (400 MHz, DMSO- d₆) δ 11.33 (s, 1H), 10.70 (s, 1H), 8.02 (s, 1H), 7.00-6.96 (m, 1H), 6.87-6.82 (m, 1H), ), 6.81-6.79 (m, 1H), 6.20 (dd, J = 16.0, 4.0 Hz, 1H), 5.75 (dd, J = 16.0, 4.0 Hz, 1H), 5.26-5.23 (m, 1H), 4.62 (s, 2H), 4.11-4.08 (m, 2H), 3.58 (t, J = 9.2, 1H), 3.46-3.43 (m, 1H), 3.40-3.34 (m, 3H), 3.28 (s, 3H), 3.01-2.97 (m, 1H), 1.47-1.34 (m, 6H). |
| 3 | 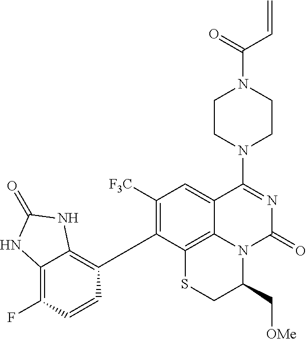 | (3S,10S)-7-(4- acryloylpiperazin-1-yl)- 10-(7-fluoro-2-oxo-2,3- dihydro-1H- benzo[d]imidazol-4-yl)- 3-(methoxymethyl)-9- (trifluoromethyl)-2,3- dihydro-5H- [1,4]thiazino[2,3,4- ij]quinazolin-5-one | 605.1 | ¹H NMR (400 MHz, DMSO- d₆) δ 7.87-7.84 (m, 1H), 6.97- 6.70 (m, 3H), 6.19-6.12 (m, 1H), 5.75-5.71 (m, 1H), 5.29- 5.16 (m, 1H), 3.97-3.87 (m, 2H), 3.77-3.67 (m, 3H), 3.58- 3.54 (m, 1H), 3.47-3.32 (m, 4H), 3.28-3.17 (m, 4H), 2.98- 2.91 (m, 1H). |
| 4 | 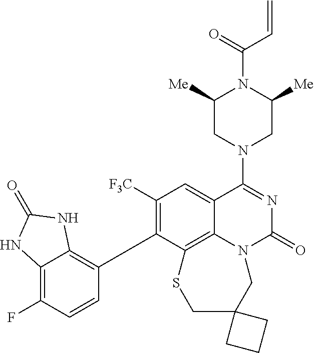 [SMILES: C=CC(=O)N1[C@H](C)CN(c2nc(=O)n3c4c(c(Cc5ccc(F)c6[nH]c(=O)[nH]c56)c(C(F)(F)F)cc24)SCC2(CCC2)C3)C[C@@H]1C] | 8′-((3S,5R)-4-acryloyl- 3,5-dimethylpiperazin- 1-yl)-11′-(7-fluoro-2- oxo-2,3-dihydro-1H- benzo[d]imidazol-4-yl)- 10′-(trifluoromethyl)- 2′H, 4′H,6′H- spiro[cyclobutane-1,3′- [1,4]thiazepino[2,3,4- ij]quinazolin]-6′-one | 643.2 | ¹H NMR (400 MHz, DMSO- d₆) δ 11.33 (s, 1H), 10.79 (s, 1H), 7.99 (s, 1H), 7.00-6.92 (m, 1H), 6.90-6.73 (m, 2H), 6.20 (dd, J = 16.0, 4.0 Hz, 1H), 5.75 (dd, J = 16.0, 4.0 Hz, 1H), 4.82-4.42 (m, 3H), 4.15-4.05 (m, 2H), 3.32-3.06 (m, 6H), 1.98-1.65 (m, 5H), 1.55-1.21 (m, 6H). |
| 5 | 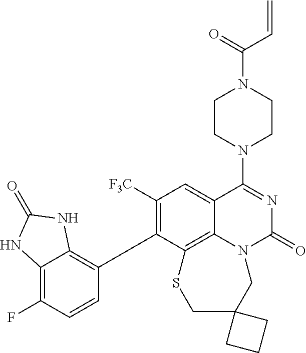 [SMILES: C=CC(=O)N1CCN(c2nc(=O)n3c4c(c(Cc5ccc(F)c6[nH]c(=O)[nH]c56)c(C(F)(F)F)cc24)SCC2(CCC2)C3)CC1] | 8′-(4-acryloylpiperazin- 1-yl)-11′-(7-fluoro-2- oxo-2,3-dihydro-1H- benzo[d]imidazol-4-yl)- 10′-(trifluoromethyl)- 2′H,4′H,6′H- spiro[cyclobutane-1,3′- [1,4]thiazepino[2,3,4- ij]quinazolin]-6′-one | 615.2 | ¹H NMR (400 MHz, DMSO- d₆) δ 11.27 (s, 1H), 10.75 (s, 1H), 7.78 (s, 1H), 6.95- 6.84 (m, 1H), 6.84-6.65 (m, 2H), 6.12 (dd, J = 16.0, 2.4 Hz, 1H), 5.68 (dd, J = 12.0, 4.0 Hz, 1H), 3.75 (m, 6H), 3.32 (s, 1H), 3.22 (m, 1H), 3.16 (m, 1H), 3.06 (d, J = 16.0 Hz, 1H), 2.49 (d, J = 16.0 Hz, 2H), 1.73 (m, 6H). |
| 6 | 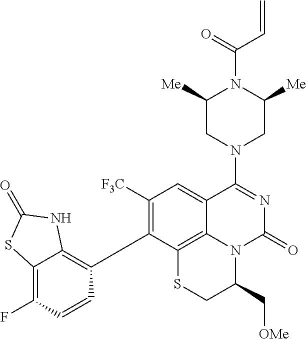 | (3S,10S)-7-((3S,5R)-4- acryloyl-3,5- dimethylpiperazin-1- yl)-10-(7-fluoro-2-oxo- 2,3- dihydrobenzo[d]thiazol- 4-yl)-3- (methoxymethyl)-9- (trifluoromethyl)-2,3- dihydro-5H- [1,4]thiazino[2,3,4- ij]quinazolin-5-one | 650 | ¹H NMR (400 MHz, DMSO- d₆) δ 11.86 (s, 1H), 8.04 (s, 1H), 7.25 (s, 1H), 7.23 (s, 1H), 6.86-6.80 (m, 1H), 6.20 (dd, J = 16.6, 2.2 Hz, 1H), 5.75 (dd, J = 10.4, 2.2 Hz, 1H), 5.29-5.28 (m, 1H), 4.63 (m, 2H), 4.11- 4.07 (d, J = 13.2 Hz, 2H), 3.59 (t, J = 9.2, 1H), 3.47-3.39 (m, 5H), 3.28 (s, 3H), 1.46 (d, J = 6.8 Hz, 3H), 1.37 (d, J = 6.8 Hz, 3H). |
| 7 | 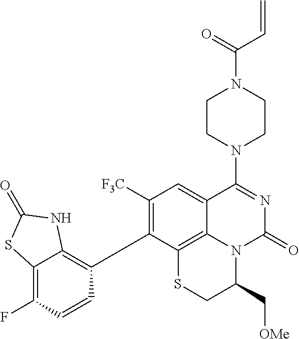 | (3S,10S)-7-(4- acryloylpiperazin-1-yl)- 10-(7-fluoro-2-oxo-2,3- dihydrobenzo[d]thiazol- 4-yl)-3- (methoxymethyl)-9- (trifluoromethyl)-2,3- dihydro-5H- [1,4]thiazino[2,3,4- ij]quinazolin-5-one | 622.1 | ¹H NMR (400 MHz, CDCl₃) δ = 9.01 (s, 1H), 7.85 (s, 1H), 7.12-7.11 (m, 1H), 7.07- 7.03 (m, 1H), 6.62-6.58 (m, 1H), 6.40-6.35 (m, 1H), 5.82- 5.79 (m, 1H), 5.40-5.39 (m, 1H), 4.01 (s, 3H), 3.84-3.81 (m, 5H), 3.65-3.61 (m, 2H), 3.39 (m, 3H), 3.34-3.30 (m, 1H), 2.95-2.92 (m, 1H). |
| 8 | 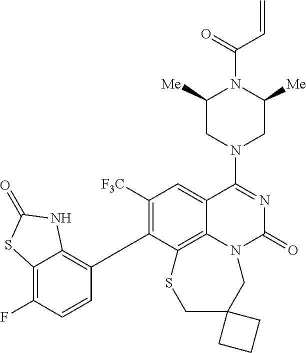 [SMILES: C=CC(=O)N1[C@H](C)CN(c2nc(=O)n3c4c(c(Cc5ccc(F)c6sc(=O)[nH]c56)c(C(F)(F)F)cc24)SCC2(CCC2)C3)C[C@@H]1C] | 8′-((3S,5R)-4-acryloyl- 3,5-dimethylpiperazin- 1-yl)-11′-(7-fluoro-2- oxo-2,3- dihydrobenzo[d]thiazol- 4-yl)-10′- (trifluoromethyl)- 2′H,4′H,6′H- spiro[cyclobutane-1,3′- [1,4]thiazepino[2,3,4- ij]quinazolin]-6′-one | 660.1 | ¹H NMR (400 MHz, CDCl₃) δ = 9.46 (s, 1H), 8.07 (s, 1H), 7.18-6.97 (m, 2H), 6.64 (dd, J = 16.7, 10.5 Hz, 1H), 6.40 (dd, J = 16.7, 1.7 Hz, 1H), 5.78 (dd, J = 10.5, 1.7 Hz, 1H), 5.10-4.32 (m, 4H), 4.29-4.06 (m, 2H), 3.53-3.28 (m, 2H), 3.24- 2.86 (m, 2H), 2.07-1.67 (m, 6H), 1.61-1.44 (m, 6H). |
| 9 | 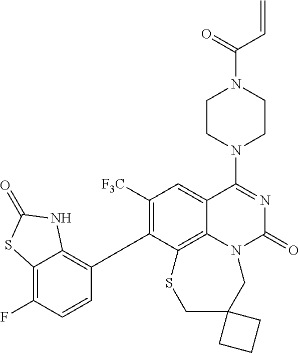 [SMILES: C=CC(=O)N1CCN(c2nc(=O)n3c4c(c(Cc5ccc(F)c6sc(=O)[nH]c56)c(C(F)(F)F)cc24)SCC2(CCC2)C3)CC1] | 8′-(4-acryloylpiperazin- 1-yl)-11′-(7-fluoro-2- oxo-2,3- dihydrobenzo[d]thiazol- 4-yl)-10′- (trifluoromethyl)- 2′H,4′H,6′H- spiro[cyclobutane-1,3′- [1,4]thiazepino[2,3,4- ij]quinazolin]-6′-one | 632.1 | ¹H NMR (400 MHz, CDCl₃) δ 7.83 (s, 1H), 7.14-6.97 (m, 2H), 6.60 (dd, J = 16.0, 8.0 Hz, 1H), 6.37 (d, J = 16.0 Hz, 1H), 5.79 (d, J = 12.0 Hz, 1H), 4.41 (s, 2H), 4.12- 3.53 (m, 8H), 3.19-2.96 (m, 2H), 2.05-1.64 (m, 6H). |
| 10 | 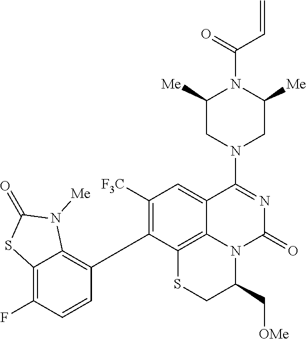 [SMILES: C=CC(=O)N1[C@H](C)CN(c2nc(=O)n3c4c(c(Cc5ccc(F)c6sc(=O)n(C)c56)c(C(F)(F)F)cc24)SC[C@@H]3COC)C[C@@H]1C] | (3S)-7-((3S,5R)-4- acryloyl-3,5- dimethylpiperazin-1- yl)-10-(7-fluoro-3- methyl-2-oxo-2,3- dihydrobenzo[d]thiazol- 4-yl)-3- (methoxymethyl)-9- (trifluoromethyl)-2,3- dihydro-5H- [1,4]thiazino[2,3,4- ij]quinazolin-5-one | 664 | ¹H NMR (400 MHz, CDCl₃) δ 8.09 (s, 1H), 7.06-6.93 (m, 2H), 6.67-6.59 (m, 1H), 6.42 (dd, J = 16.4, 1.6, 1H), 5.79 (dd, J = 10.4, 1.6, 1H), 5.47 (m, 1H), 4.69 (m, 2H), 4.18 (t, J = 15.2, 2H), 3.64 (d, J = 7.2, 2H), 3.49-3.32 (m, 6H), 3.07-2.92 (m, 4H), 1.62 (d, J = 6.8, 3H), 1.49 (d, J = 6.8, 3H). |

### Example 1: (3S)-8-((3S,5R)-4-acryloyl-3,5-dimethylpiperazin-1-yl)-11-(7-fluoro-2-oxo-2,3-dihydro-1H-benzo[d]imidazol-4-yl)-3-methoxy-10-(trifluoromethyl)-3,4-dihydro-2H,6H-[1,4]thiazepino[2,3,4-ij]quinazolin-6-one

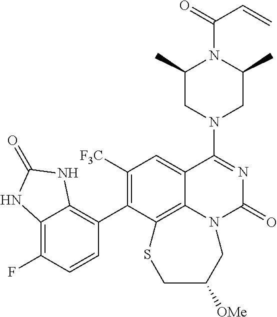 [SMILES: C=CC(=O)N1[C@H](C)CN(c2nc(=O)n3c4c(c(-c5ccc(F)c6[nH]c(=O)[nH]c56)c(C(F)(F)F)cc24)SC[C@@H](OC)C3)C[C@@H]1C]

To a mixture of (3S)-8-((3S,5R)-3,5-dimethylpiperazin-1-yl)-11-(7-fluoro-2-oxo-2,3-dihydro-1H-benzo[d]imidazol-4-yl)-3-methoxy-10-(trifluoromethyl)-3,4-dihydro-2H,6H-[1,4]thiazepino[2,3,4-ij]quinazolin-6-one (70 mg) and triethylamine (0.446 mmol) in dichloromethane (3 mL) was added prop-2-enoyl chloride (0.166 mmol). The reaction mixture was stirred for 1 hour, concentrated and the resulting crude material purified by HPLC. The title compound was isolated in 27% yield as a white solid.

MS (ESI) m/z=633.1 [M+H]+

¹H NMR (400 MHz, DMSO-d₆) δ11.35 (s, 1H), 10.85 (s, 1H), 8.03-7.99 (m, 1H), 6.98-6.94 (m, 1H), 6.88-6.72 (m, 2H), 6.20 (dd, J=16.0, 4.0 Hz, 1H), 5.75 (dd, J=8.0, 4.0 Hz, 1H), 4.61-4.55 (m, 3H), 4.11-4.05 (m, 2H), 3.86-3.81 (m, 1H), 3.34-3.30 (m, 7H), 3.14 (m, 1H), 1.43-1.38 (m, 6H).

### Step 1: 4-Bromo-7-fluoro-1,3-dihydro-2H-benzo[d]imidazol-2-one

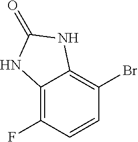 [SMILES: O=c1[nH]c2c(F)ccc(Br)c2[nH]1]

To a solution of 2-amino-3-bromo-6-fluorobenzoic acid (8.54 mmol) and triethylamine (25.7 mmol) in dioxane (20 mL) was added DPPA (9.0 mmol) and the mixture was stirred at 100° C. for 2 hours. Evaporation of volatiles under reduced pressure afforded a residue that was purified by reverse phase chromatography (5-60% acetonitrile in water with 0.10% TFA). The title compound was isolated in 81% yield as a gray solid.

MS (ESI) m/z=231.0/232.9 [M+H]+

### Step 2: 4-fluoro-7-(4,4,5,5-tetramethyl-1,3,2-dioxaborolan-2-yl)-1,3-dihydro-2H-benzo[d]imidazol-2-one

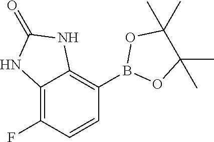 [SMILES: CC1(C)OB(c2ccc(F)c3[nH]c(=O)[nH]c23)OC1(C)C]

A mixture of 4-bromo-7-fluoro-1,3-dihydro-2H-benzo[d]imidazol-2-one (1.3 mmol), 4,4,4′,4′,5,5,5′,5′-octamethyl-2,2′-bi(1,3,2-dioxaborolane) (1.82 mmol), Pd(dppf)Cl₂ (0.26 mmol) and KOAc (3.89 mmol) in DMF (8 mL) was stirred at 100° C. for 1 hour. The mixture was concentrated to afford a residue that was purified by reversed phase chromatography (1-50% acetonitrile in water with 0.1% TFA). The title compound was isolated in 89% yield as a yellow solid.

MS (ESI) m/z=279.1 [M+H]+

### Step 3: 5-Chloro-2-iodo-4-(trifluoromethyl)aniline

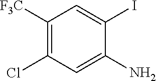 [SMILES: Nc1cc(Cl)c(C(F)(F)F)cc1I]

To a solution of 3-chloro-4-(trifluoromethyl)aniline (0.13 mol) in acetic acid (270 mL) was added NaIO₄ (0.13 mol) and NaCl (0.26 mol). The reaction was stirred for 5 minutes and then a solution of potassium iodide (0.13 mol) in H₂O (30 mL) was added and the mixture heated at 50° C. for 16 hours. The reaction was cooled down to room temperature, diluted with water, extracted with ethyl acetate three times and the combined organic layers washed with saturated aqueous Na₂S₂O₃ and brine and dried over Na₂SO₄. The solids were filtered and the volatiles removed under reduced pressure to afford the title compound in 82% yield as a brown solid.

MS (ESI) m/z=321.9 [M+H]+

### Step 4: 2-Amino-4-chloro-5-(trifluoromethyl)benzonitrile

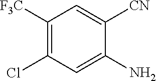 [SMILES: N#Cc1cc(C(F)(F)F)c(Cl)cc1N]

To a mixture of 5-chloro-2-iodo-4-(trifluoromethyl)aniline (0.16 mol) in DMF (500 ml) was added CuCN (0.32 mol) and the mixture was stirred at 110° C. for 20 hours. After being cooled down to room temperature, the solids were filtered and the solution diluted with water and extracted with ethyl acetate three times. The combined organic layers were washed with brine, dried over Na₂SO₄, filtered and concentrated under reduced pressure to afford a residue that was purified by flash chromatography (0-15% ethyl acetate in hexanes). The title compound was isolated in 80% yield as a gray solid.

MS (ESI) m/z=221.0 [M+H]+

### Step 5: 2-amino-4-chloro-3-iodo-5-(trifluoromethyl)benzonitrile

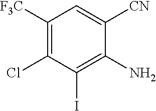 [SMILES: N#Cc1cc(C(F)(F)F)c(Cl)c(I)c1N]

To a mixture of 2-amino-4-chloro-5-(trifluoromethyl)benzonitrile (0.045 mol) in acetic acid (300 mL) were added NaIO₄ (0.031 mol), sulfuric acid (0.36 mol) and potassium iodide (0.046 mol). The mixture was stirred at 80° C. for 16 hours, cooled down to room temperature, diluted with water and extracted with ethyl acetate three times. The combined organic layers were washed with saturated aqueous Na₂S₂O₃ and brine, dried over sodium sulfate and filtered. Evaporation of volatiles under reduced pressure afforded a crude product that was triturated with hexanes and ethyl acetate (105 mL, 20/1). The title compound was isolated in 77% yield as a yellow solid.

MS (ESI) m/z=346.9 [M+H]+

### Step 6: 7-chloro-8-iodo-6-(trifluoromethyl)quinazoline-2,4(1H,3H)-dione

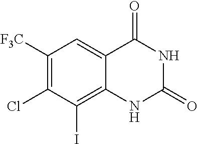 [SMILES: O=c1[nH]c(=O)c2cc(C(F)(F)F)c(Cl)c(I)c2[nH]1]

To a solution of 2-amino-4-chloro-5-[(difluoromethyl)-$1\hat{ }{2}-fluoranyl]-3-iodobenzonitrile (0.022 mol) in DMF (75 mL) was added DBU (0.066 mol) and the reaction was stirred for 16 hours under CO₂. The mixture was diluted with water and extracted with ethyl acetate. The organic layer was washed with brine, dried over sodium sulfate, filtered and concentrated under reduced pressure to afford a crude material that was purified by C18 chromatography (0-95% acetonitrile-water) to a afford the title compound in 50% yield as a yellow solid.

Mass Spectrum (ESI) m/z=390.9 [M+H]+

¹H NMR (400 MHz, DMSO-d₆) δ=11.90 (s, 1H), 9.97 (s, 1H), 8.20 (s, 1H).

### Step 7: tert-butyl (2S,6R)-4-((3S)-11-(7-fluoro-2-oxo-2,3-dihydro-1H-benzo[d]imidazol-4-yl)-3-methoxy-6-oxo-10-(trifluoromethyl)-3,4-dihydro-2H,6H-[1,4]thiazepino[2,3,4-ij]quinazolin-8-yl)-2,6-dimethylpiperazine-1-carboxylate

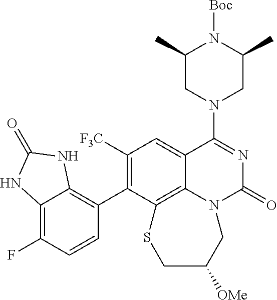 [SMILES: CO[C@@H]1CSc2c(-c3ccc(F)c4[nH]c(=O)[nH]c34)c(C(F)(F)F)cc3c(N4C[C@@H](C)N(C(=O)OC(C)(C)C)[C@@H](C)C4)nc(=O)n(c23)C1]

A mixture of tert-butyl (2S,6R)-4-((S)-11-chloro-3-methoxy-6-oxo-10-(trifluoromethyl)-3,4-dihydro-2H,6H-[1,4]thiazepino[2,3,4-ij]quinazolin-8-yl)-2,6-dimethylpiperazine-1-carboxylate (0.15 mmol), 4-fluoro-7-(4,4,5,5-tetramethyl-1,3,2-dioxaborolan-2-yl)-1,3-dihydro-2H-benzo[d]imidazol-2-one (0.37 mmol), RuPhos Pd G2 (0.018 mmol) and K₃PO₄ (0.528 mmol) in dioxane/H₂O (4 mL/1 mL) was stirred at 80° C. for 2 hours. The volatiles were removed under reduced pressure and the residue was purified by preparative HPLC. The title compound was isolated in 110% yield as a white solid.

MS (ESI) m/z=679.1 [M+H]+

### Step 8: (3S)-8-((3S,5R)-3,5-dimethylpiperazin-1-yl)-11-(7-fluoro-2-oxo-2,3-dihydro-1H-benzo[d]imidazol-4-yl)-3-methoxy-10-(trifluoromethyl)-3,4-dihydro-2H,6H-[1,4]thiazepino[2,3,4-ij]quinazolin-6-one

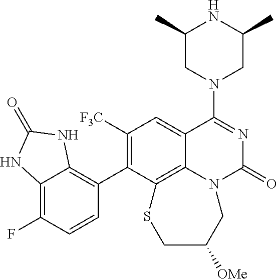 [SMILES: CO[C@@H]1CSc2c(-c3ccc(F)c4[nH]c(=O)[nH]c34)c(C(F)(F)F)cc3c(N4C[C@@H](C)N[C@@H](C)C4)nc(=O)n(c23)C1]

To a 0° C. solution of tert-butyl (2S,6R)-4-((3S)-11-(7-fluoro-2-oxo-2,3-dihydro-1H-benzo[d]imidazol-4-yl)-3-methoxy-6-oxo-10-(trifluoromethyl)-3,4-dihydro-2H,6H-[1,4]thiazepino[2,3,4-ij]quinazolin-8-yl)-2,6-dimethylpiperazine-1-carboxylate (0.094 mmol) in dichloromethane (4 mL) was added TFA (1 mL). After 1 hour the reaction mixture was concentrated under reduced pressure to afford 70 mg of crude title compound that was used in the next step without further purification.

MS (ESI) m z=579.1 (M+H)⁺.

### Example 7: 3S,10S)-7-(4-acryloylpiperazin-1-yl)-10-(7-fluoro-2-oxo-2,3-dihydrobenzo[d]thiazol-4-yl)-3-(methoxymethyl)-9-(trifluoromethyl)-2,3-dihydro-5H-[1,4]thiazino[2,3,4-ij]quinazolin-5-one

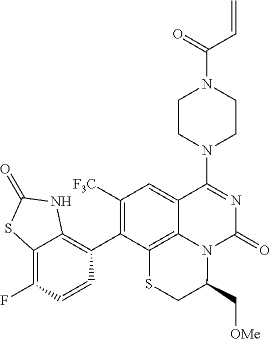

The title compound was prepared analogously to Example 1, where tert-butyl (2S,6R)-4-((S)-11-chloro-3-methoxy-6-oxo-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (S)-4-(10-chloro-3-(methoxymethyl)-5-oxo-9-(trifluoromethyl)-2,3-dihydro-5H-[1,4]thiazino[2,3,4-ij]quinazolin-7-yl)piperazine-1-carboxylate. MS (ESI) m/z=622.1 [M+H]+.

¹H NMR (400 MHz, CDCl₃) δ=9.01 (s, 1H), 7.85 (s, 1H), 7.12-7.11 (m, 1H), 7.07-7.03 (m, 1H), 6.62-6.58 (m, 1H), 6.40-6.35 (m, 1H), 5.82-5.79 (m, 1H), 5.40-5.39 (m, 1H), 4.01 (s, 3H), 3.84-3.81 (m, 5H), 3.65-3.61 (m, 2H), 3.39 (m, 3H), 3.34-3.30 (m, 1H), 2.95-2.92 (m, 1H).

### Example 10: (3S)-7-((3S,5R)-4-acryloyl-3,5-dimethylpiperazin-1-yl)-10-(7-fluoro-3-methyl-2-oxo-2,3-dihydrobenzo[d]thiazol-4-yl)-3-(methoxymethyl)-9-(trifluoromethyl)-2,3-dihydro-5H-[1,4]thiazino[2,3,4-ij]quinazolin-5-one

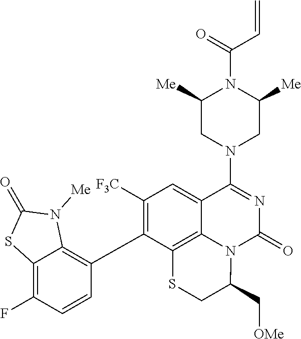 [SMILES: C=CC(=O)N1[C@H](C)CN(c2nc(=O)n3c4c(c(-c5ccc(F)c6sc(=O)n(C)c56)c(C(F)(F)F)cc24)SC[C@@H]3COC)C[C@@H]1C]

### Step 1: tert-butyl (2S,6R)-4-((3S)-10-(7-fluoro-3-methyl-2-oxo-2,3-dihydrobenzo[d]thiazol-4-yl)-3-(methoxymethyl)-5-oxo-9-(trifluoromethyl)-2,3-dihydro-5H-[1,4]thiazino[2,3,4-ij]quinazolin-7-yl)-2,6-dimethylpiperazine-1-carboxylate

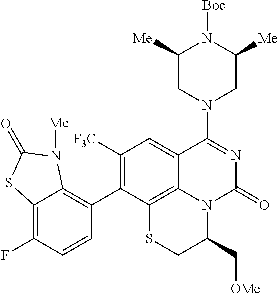 [SMILES: COC[C@H]1CSc2c(-c3ccc(F)c4sc(=O)n(C)c34)c(C(F)(F)F)cc3c(N4C[C@@H](C)N(C(=O)OC(C)(C)C)[C@@H](C)C4)nc(=O)n1c23]

To a solution of tert-butyl (2S,6R)-4-((3S)-10-(7-fluoro-2-oxo-2,3-dihydrobenzo[d]thiazol-4-yl)-3-(methoxymethyl)-5-oxo-9-(trifluoromethyl)-2,3-dihydro-5H-[1,4]thiazino[2,3,4-ij]quinazolin-7-yl)-2,6-dimethylpiperazine-1-carboxylate (0.43 mmol) and NaOH (1.30 mmol) in THF:H2O (1:5 mL) was added dimethyl sulfate (1.42 mmol). The solution was stirred at room temperature for 1 hour, diluted with ethyl acetate, washed with brine and the organic layer dried over sodium sulfate. Filtration and evaporation of volatiles afforded a residue that was purified by reverse phase chromatography (50-75% acetonitrile in water, containing 0.1% TFA as additive) to afford the title compound in 28% yield as a yellow solid. Mass Spectrum (ESI) m/z=710.1 (M+H)+.

### Step 2: (3S)-7-((3S,5R)-3,5-dimethylpiperazin-1-yl)-10-(7-fluoro-3-methyl-2-oxo-2,3-dihydrobenzo[d]thiazol-4-yl)-3-(methoxymethyl)-9-(trifluoromethyl)-2,3-dihydro-5H-[1,4]thiazino[2,3,4-ij]quinazolin-5-one

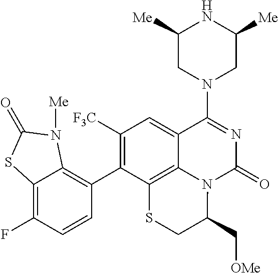 [SMILES: COC[C@H]1CSc2c(-c3ccc(F)c4sc(=O)n(C)c34)c(C(F)(F)F)cc3c(N4C[C@@H](C)N[C@@H](C)C4)nc(=O)n1c23]

The title compound was prepared analogously to Example 1, step 8, where tert-butyl (2S,6R)-4-((3S)-11-(7-fluoro-2-oxo-2,3-dihydro-1H-benzo[d]imidazol-4-yl)-3-methoxy-6-oxo-10-(trifluoromethyl)-3,4-dihydro-2H,6H-[1,4]thiazepino[2,3,4-ij]quinazolin-8-yl)-2,6-dimethylpiperazine-1-carboxylate was replaced with tert-butyl (2S,6R)-4-((3S)-10-(7-fluoro-3-methyl-2-oxo-2,3-dihydrobenzo[d]thiazol-4-yl)-3-(methoxymethyl)-5-oxo-9-(trifluoromethyl)-2,3-dihydro-5H-[1,4]thiazino[2,3,4-ij]quinazolin-7-yl)-2,6-dimethylpiperazine-1-carboxylate. MS (ESI) m/z=610.1 [M+H]+.

### Step 3: (3S)-7-((3S,5R)-4-acryloyl-3,5-dimethylpiperazin-1-yl)-10-(7-fluoro-3-methyl-2-oxo-2,3-dihydrobenzo[d]thiazol-4-yl)-3-(methoxymethyl)-9-(trifluoromethyl)-2,3-dihydro-5H-[1,4]thiazino[2,3,4-ij]quinazolin-5-one

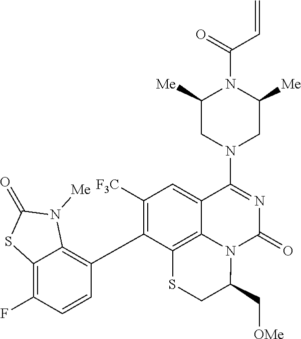 [SMILES: C=CC(=O)N1[C@H](C)CN(c2nc(=O)n3c4c(c(-c5ccc(F)c6sc(=O)n(C)c56)c(C(F)(F)F)cc24)SC[C@@H]3COC)C[C@@H]1C]

The title compound was prepared analogously to Example 1, where (3S)-8-((3S,5R)-3,5-dimethylpiperazin-1-yl)-11-(7-fluoro-2-oxo-2,3-dihydro-1H-benzo[d]imidazol-4-yl)-3-methoxy-10-(trifluoromethyl)-3,4-dihydro-2H,6H-[1,4]thiazepino[2,3,4-ij]quinazolin-6-one was replaced with (3S)-7-((3S,5R)-3,5-dimethylpiperazin-1-yl)-10-(7-fluoro-3-methyl-2-oxo-2,3-dihydrobenzo[d]thiazol-4-yl)-3-(methoxymethyl)-9-(trifluoromethyl)-2,3-dihydro-5H-[1,4]thiazino[2,3,4-ij]quinazolin-5-one. MS (ESI) m/z=664.0 [M+H]+.

¹H NMR (400 MHz, CDCl₃) δ8.09 (s, 1H), 7.06-6.93 (m, 2H), 6.67-6.59 (m, 1H), 6.42 (dd, J=16.4, 1.6, 1H), 5.79 (dd, J=10.4, 1.6, 1H), 5.47 (m, 1H), 4.69 (m, 2H), 4.18 (t, J=15.2, 2H), 3.64 (d, J=7.2, 2H), 3.49-3.32 (m, 6H), 3.07-2.92 (m, 4H), 1.62 (d, J=6.8, 3H), 1.49 (d, J=6.8, 3H).

### Assays & Activity Data

KRAS G12C covalent binding assays were carried out as follows:

### KRAS G12C Covalent Adduct Formation (CAF) Assay

This Example provides a protocol for assessing covalent adduct formation (CAF) between the compounds of Formula (I) and KRAS.

In vitro covalent adduct formation assay: Covalent adduct formation (CAF) reactions between Cys12 of the KRAS 4B G12C protein and the compounds disclosed herein were measured in vitro using liquid chromatography-mass spectrometry (LC-MS).

Recombinant Human KRAS 4B protein containing the G12C mutation was used in compound screening experiments. This protein contained 188 amino acids in total, including an N-terminal 6-Histidine tag, followed by a Tobacco Etch Virus (TEV) tag, followed by residues 1-169 of the native KRAS 4B sequence. The exact mass of the protein is 21,310 Da as determined by mass spectrometry. The full amino acid sequence is shown below:

(SEQ. ID NO.: 1)
MAHHHHHHAG GAENLYFQSM TEYKLVVVGA CGVGKSALTI
QLIQNHFVDE YDPTIEDSYR KQVVIDGETC LLDILDTAGQ
EEYSAMRDQY MRTGEGFLCV FAINNTKSFE DIHHYREQIK
RVKDSEDVPM VLVGNKCDLP SRTVDTKQAQ DLARSYGIPF
IETSAKTRQG VDDAFYTLVR EIRKHKEK

In an alternative screen, the assay was conducted using a KRAS 4b G12C protein having 170 amino acids, a mass of 19,336 Da, and the amino acid sequence

(SEQ. ID NO.: 2)
SMTEYKLVVVGA CGVGKSALTI QLIQNHFVDE YDPTIEDSYR
KQVVIDGETC LLDILDTAGQ EEYSAMRDQY MRTGEGFLCV
FAINNTKSFE DIHHYREQIK RVKDSEDVPM VLVGNKCDLP
SRTVDTKQAQ DLARSYGIPF IETSAKTRQG VDDAFYTLVR
EIRKHKEK.

The recombinant protein is expressed in E. coli BL21 cells and purified using affinity chromatography via a Ni-NTA column. Protein stocks were nucleotide-exchanged to >95% GDP, concentrated to 4 mg/mL, and stored at −80° C. in storage buffer (50 mM HEPES pH 7.4, 50 mM NaCl, 5 mM MgCl₂, 1 mM DTT). Pure KRAS 4B G12C protein is diluted to a concentration of 5 μM in Tris Buffered Saline, pH 7.4. The compounds were dissolved in DMSO and added to the diluted protein to make a 10 μM concentration. The total DMSO concentration in the reaction was 4%. The reaction was mixed by pipetting and incubated at 22° C. for one hour. Aliquots of the reaction were taken over time and diluted 2:1 in 0.1% formic acid. The intact mass of the protein samples was measured by LC-MS using a QExactive+ mass spectrometer (Thermo Scientific). An amount of 500 ng total protein was injected onto a C8 reverse phase column, eluted with a seven-minute gradient of 30%-90% acetonitrile/0.1% formic acid, and analyzed for intact mass by the mass spectrometer. Adducts identified were confirmed to be within 1 Dalton of the expected mass, and the relative ratios of free:adduct protein were used to quantify the percentage of protein bound by the compound. CAF reactions were run in duplicate, with a typical variability of ±5%.

Examples were evaluated in the above CAF assay at 60 minutes.

| Example number | % CAF @ 60 min |
|---|---|
| Example 1* | 75% |

*mixture of atropisomers

### Inhibition of KRAS G12C-Mediated Phosphor-ERK1/2 Inhibition by Exemplary Compounds

KRAS G12C mutant lines, NCI H358 (ATCC, CRL-5807), and Ras Initiative (RI) KRAS G12C were cultured according to published protocols and maintained at 37° C. in 5% CO₂. The phospho-ERK HTRF assay was executed following provider's protocol (CisBio #64AERPEH). NCI-H358 or RI KRAS G12C cells were plated at a density of 50,000 cells per well in a 96-well plate (Corning #3903) in respective medias (for NCI-H358, RPMI+10% FBS+1% Pen/Strep, and for RI KRAS G12C, DMEM+10% FBS+1% Pen/Strep+4 μg/mL Blasticidin) and maintained at 37° C. in 5% CO₂. Cells were allowed to adhere overnight and treated the following day with a Tecan D300e Digital Dispenser (Tecan Group Ltd., Switzerland) using an 11-point dose response starting at 2,500 nM of exemplified compounds followed by sequential 1:3 dilutions for either 4 hours or 16 hours. Following compound treatment, the cells were washed once with ice-cold PBS. Cells were lysed by adding 50 μL of lysis buffer (1×) supplemented with 1× Pierce Halt Protease and Phosphatase inhibitor and incubated for 30 minutes at 4° C. with shaking. After lysis, 16 μL of cell lysate from the 96-well cell-culture plate was transferred to a 384-well plate (Perkin Elmer #6007290). The premixed antibody solution was prepared by mixing (vol/vol) advanced phospho-ERK1/2 d2 antibody and advanced phospho-ERK1/2 Eu Cryptate antibody. The premixed antibody solution (4 μL) was added to the detection plate containing cell lysate. The detection plate was incubated overnight at 4° C., the HTRF signal was read the next day by using either a Spectramax M5 or Spectramax i3 microplate reader (Molecular Devices, San Jose, CA, USA), and data was processed according to manufacturer's protocol.

Examples were evaluated according to the above p-ERK assay

| Example No. | pERK (RI) |
|---|---|
| 1 | >2,500 |
| 2 | >2,500 |
| 3 | >2,500 |
| 4 | >2,500 |
| 5 | >2,500 |
| 6 | 68 |
| 7 | 18 |
| 8 | 171 |
| 9 | 222 |
| 10 | 46 |

Although the foregoing embodiments have been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound, or a pharmaceutically acceptable salt thereof, of Formula (I): 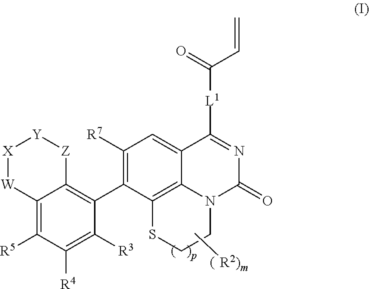
wherein
wherein W, X, Y, and Z are independently selected from N, NH, CH, C═O, C═S, CNH₂, CNHMe, S or null; wherein null can only occur once, and at least one of W, X, Y, and Z is NH and at least one of W, X, Y, and Z adjacent to the NH is C═O;
m is 1 or 2;
p is 1 or 2;
L¹ is 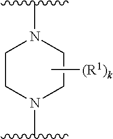
wherein k is an integer from 0 to 4; and each R¹ is independently selected from methyl, and cyanomethyl, C₂-C₄ alkyl, cyano, cycloalkyl, halo, haloalkyl, trifluoromethyl, and alkoxy; or any two R¹ combine to form a fused ring, bridge or spirocycle structure optionally comprising a heteroatom in the bridge or spirocycle selected from S, SO₂, O or N, and wherein the bridge or spirocycle structure is optionally substituted with oxo;
each R² is independently selected from the group consisting of alkyl, N-alkylamino, N, N-dialkylamino, alkylamidoalkyl, arylamidoalkyl, —OCH₂CONRR′, heteroarylalkoxyalkyl, and heteroaryloxy, wherein R and R′ are independently selected from hydrogen, alkyl, and cycloalkyl, alkylsulfonamidoalkyl, arylsulfonamidoalkyl, N-alkyl aminoalkyl, N,N-dialkyl aminoalkyl, alkoxy, alkoxyalkyl, cycloalkyl, alkylcycloalkyl, hydroxyalkyl, halogen, haloalkyl, aryl, aryloxy, aralkyl, heteroaryl, heteroarylalkyl, heterocyclyl, heterocyclylalkyl, and heteroaryloxy any of which are optionally substituted; or when m is 2, two R² combine to form a spirocyclic 3-6-membered ring optionally containing 1 to 3 heteroatoms selected from N, O, or S;
R³, R⁴, and R⁵, are independently selected from halogen, hydrogen, hydroxyl, alkoxy, alkyl, cycloalkyl, amino, N-alkylamino;
R⁶ is H or methyl; and
R⁷ is alkyl, cyano, cycloalkyl, halogen, haloalkyl, trifluoromethyl, and alkoxy.

2. The compound of claim 1, wherein n is 1.

3. The compound of claim 1, wherein n is 2.

4. The compound of claim 1, wherein n is 3.

5. The compound of claim 1, wherein n is 4.

6. The compound of any one of claims 1 to 5, wherein p is 1.

7. The compound of any one of claims 1 to 5, wherein p is 2.

8. The compound of claim 1, wherein R³ is halogen, R⁴ is hydrogen and R⁵ is hydrogen.

9. The compound of claim 1, wherein R⁵ is halogen, R⁴ is hydrogen and R³ is hydrogen.

10. The compound of claim 1, wherein R³ and R⁵ are each independently a halogen and R⁴ is hydrogen.

11. The compound of any one of claims 1 to 10, wherein L¹ selected from: 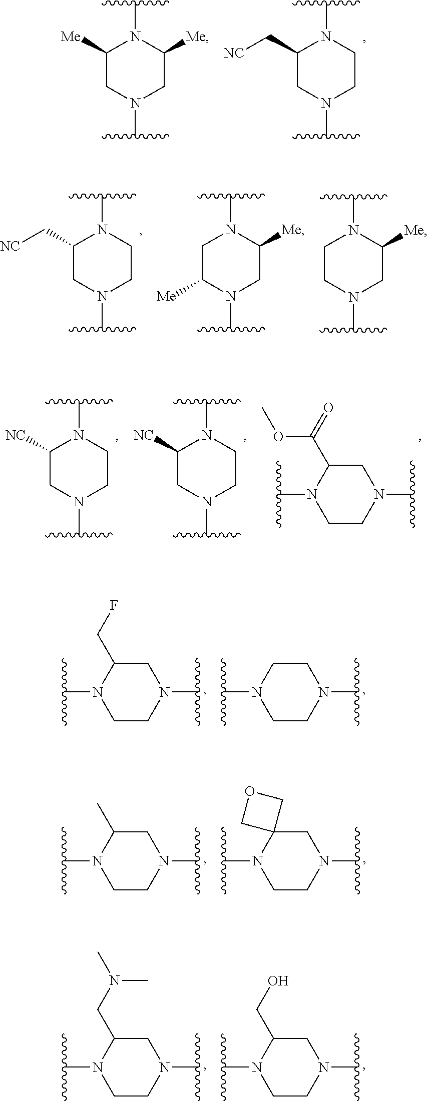 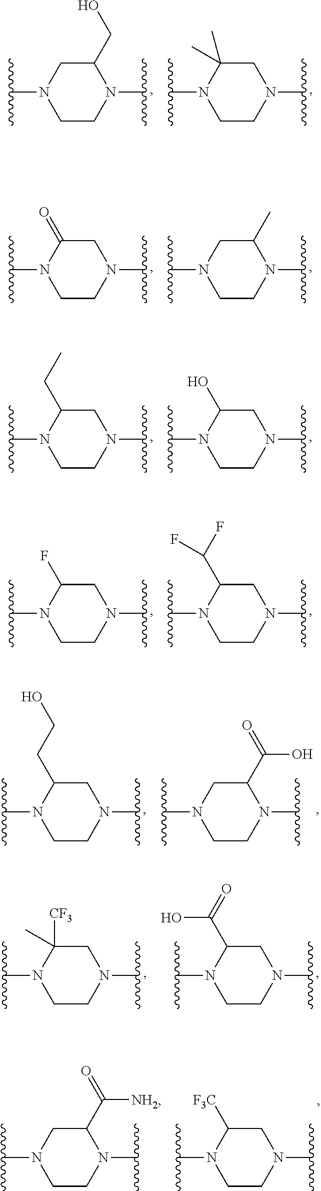 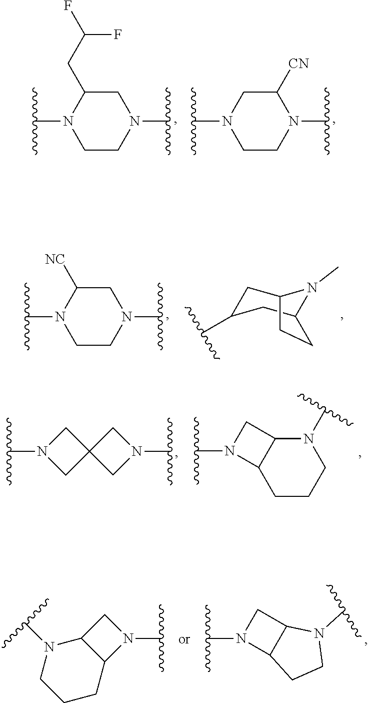
wherein 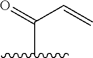 [SMILES: *C(=O)C=C]
is attached through either of the two nitrogen atoms of L¹.

12. The compound of any one of claims 1 to 9, wherein R² is selected from methoxy, amino, MeOCH₂—, EtOCH₂-, MeO(CH₂)₂NH—, 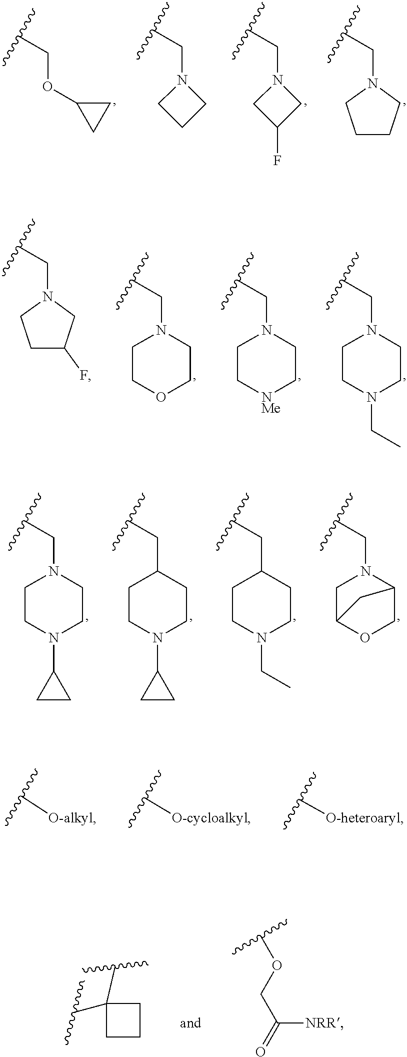
wherein R and R′ are independently selected from hydrogen, alkyl, and cycloalkyl.

13. The compound of any one of claims 1 to 12, wherein W, X, Y, and Z define a heterocycle selected from:

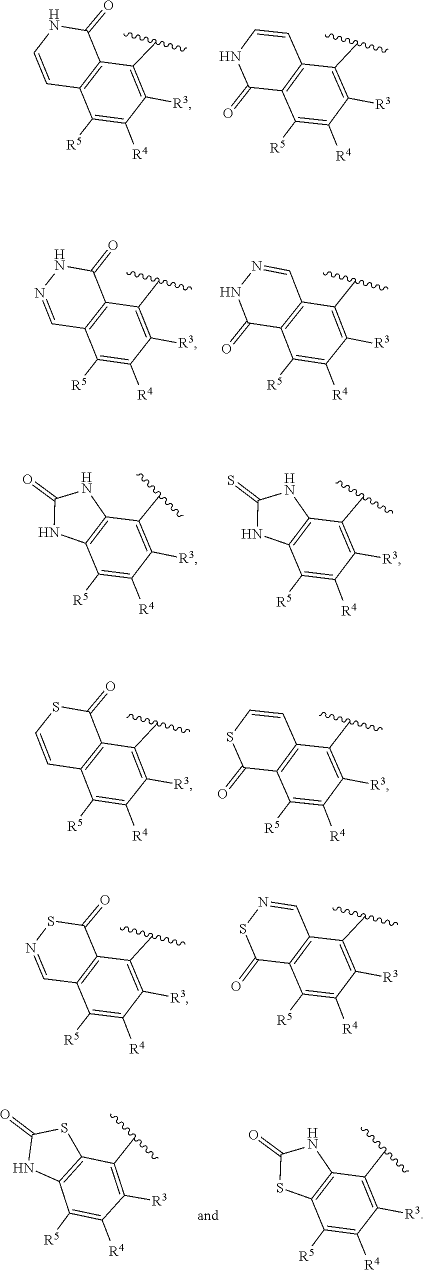

14. The compound of any one of claims 1 to 12, wherein X and Y define the heterocycle:

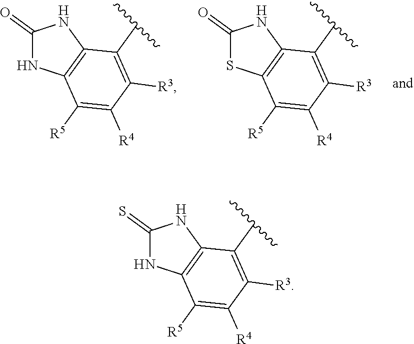

15. The compound of claim 1, wherein the compound is: 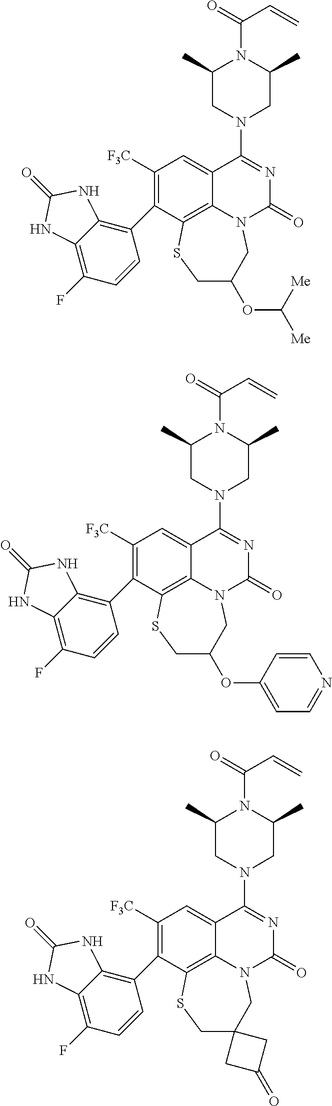 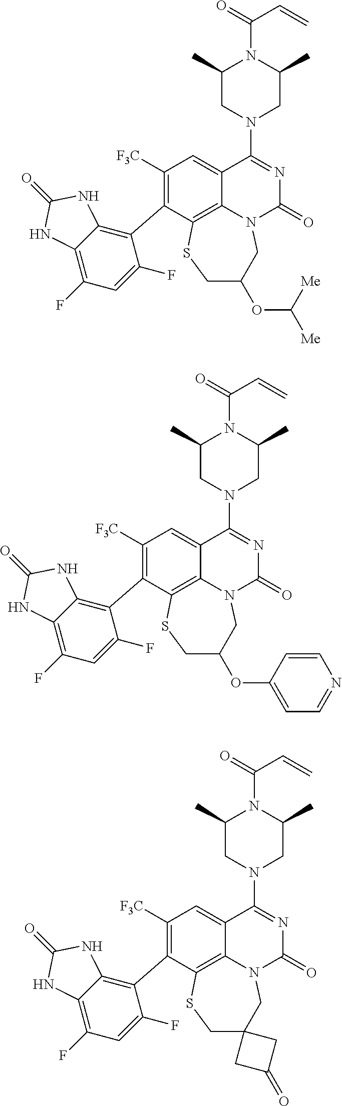 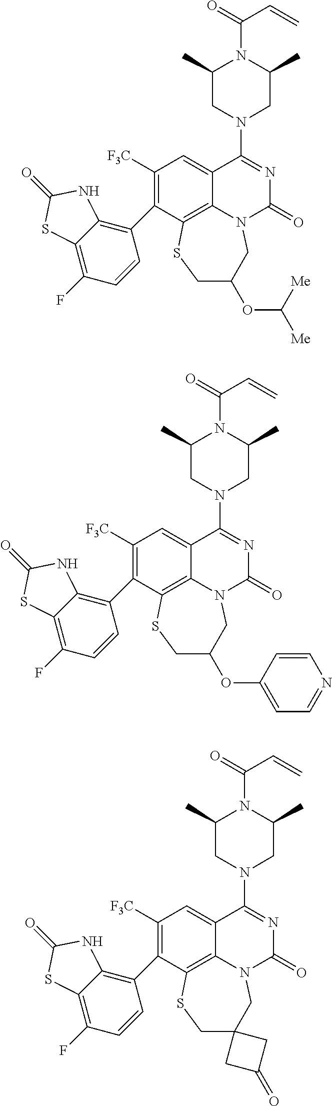 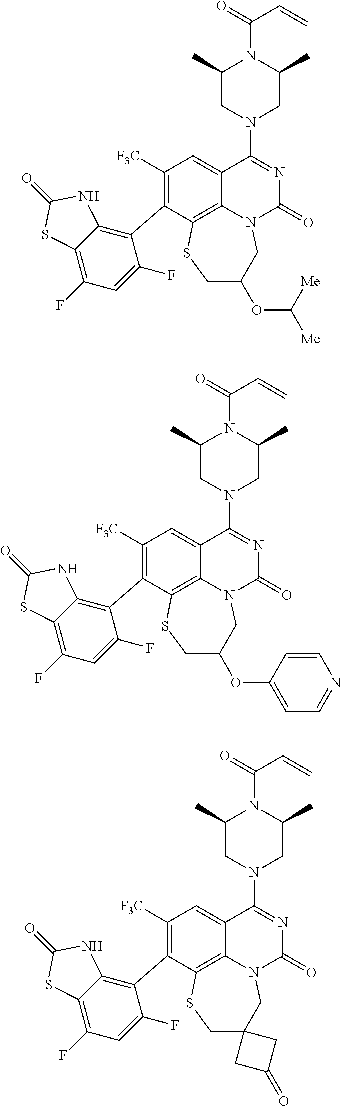 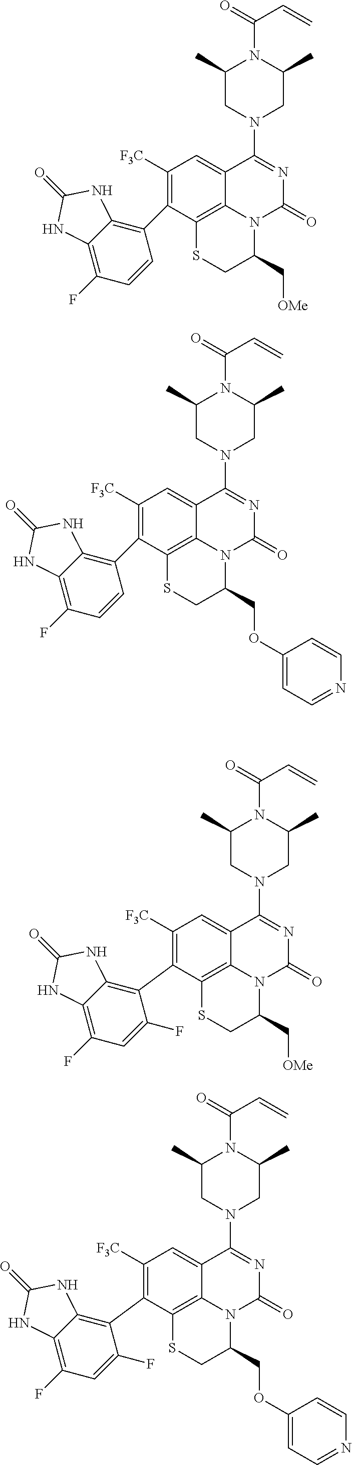 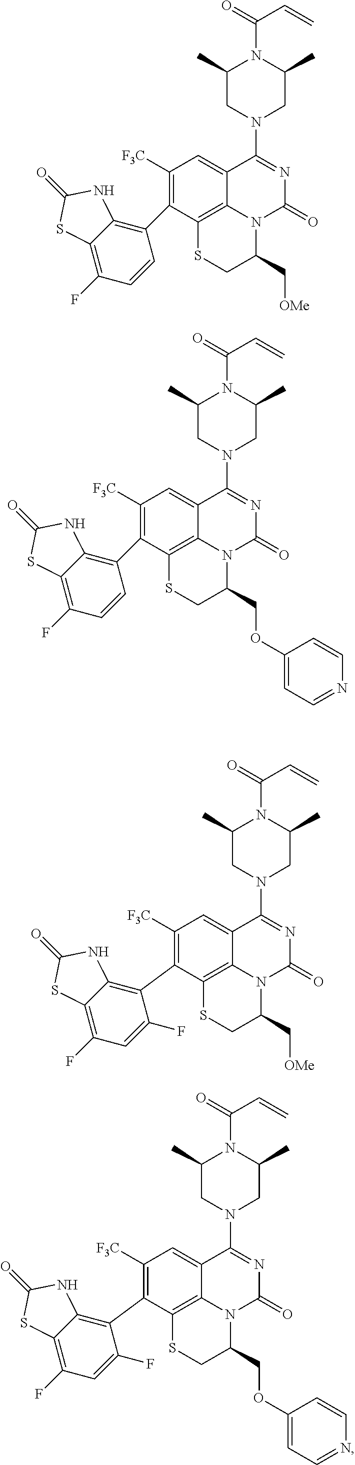 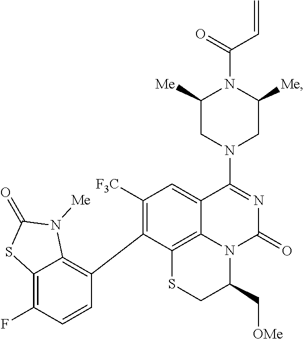 [SMILES: C=CC(=O)N1[C@H](C)CN(c2nc(=O)n3c4c(c(-c5ccc(F)c6sc(=O)n(C)c56)c(C(F)(F)F)cc24)SC[C@@H]3COC)C[C@@H]1C] 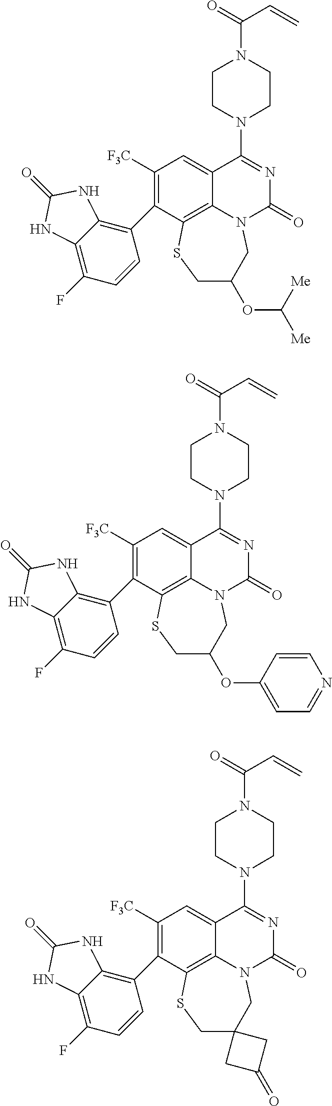 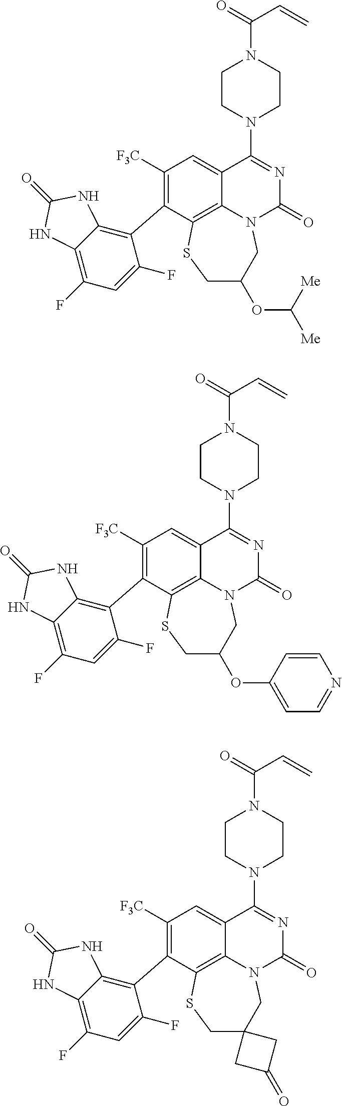 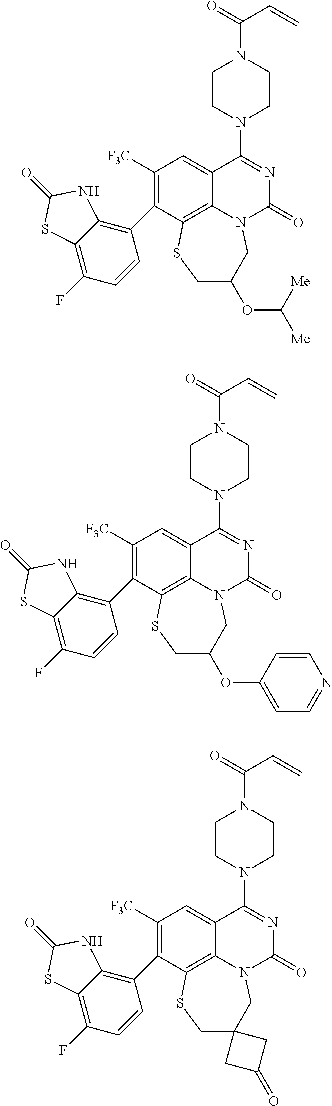 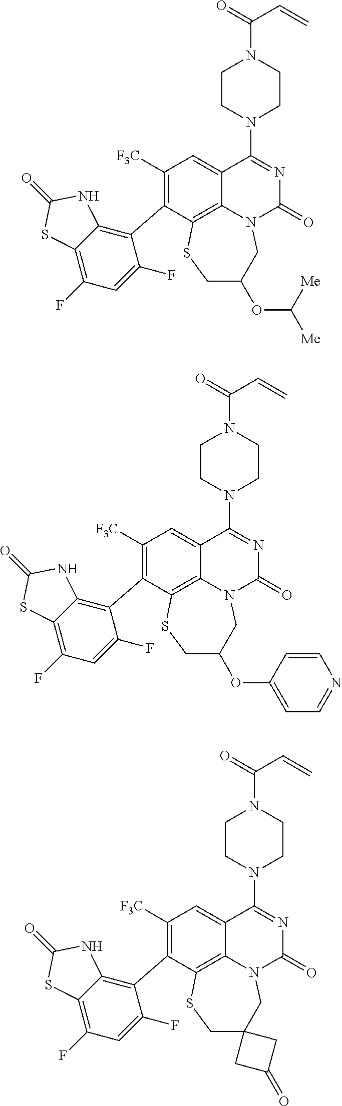 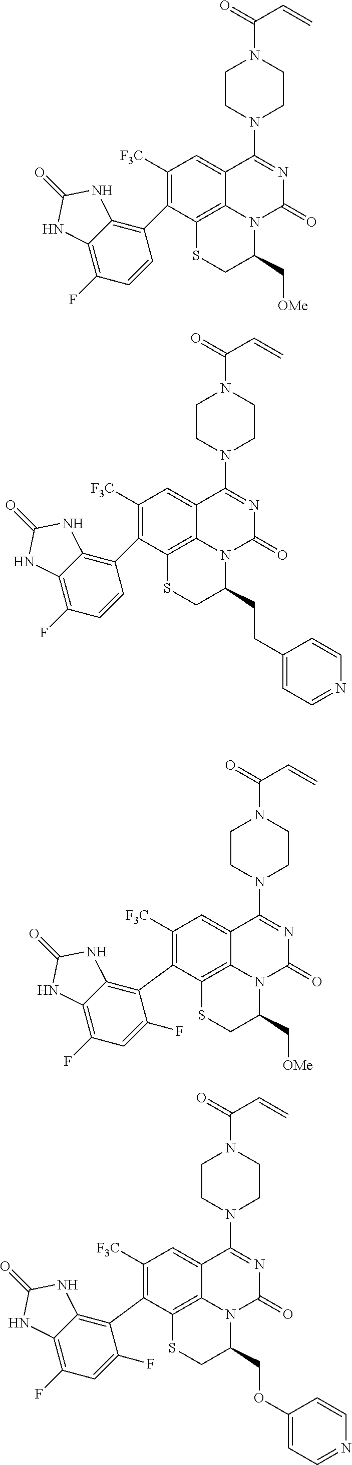 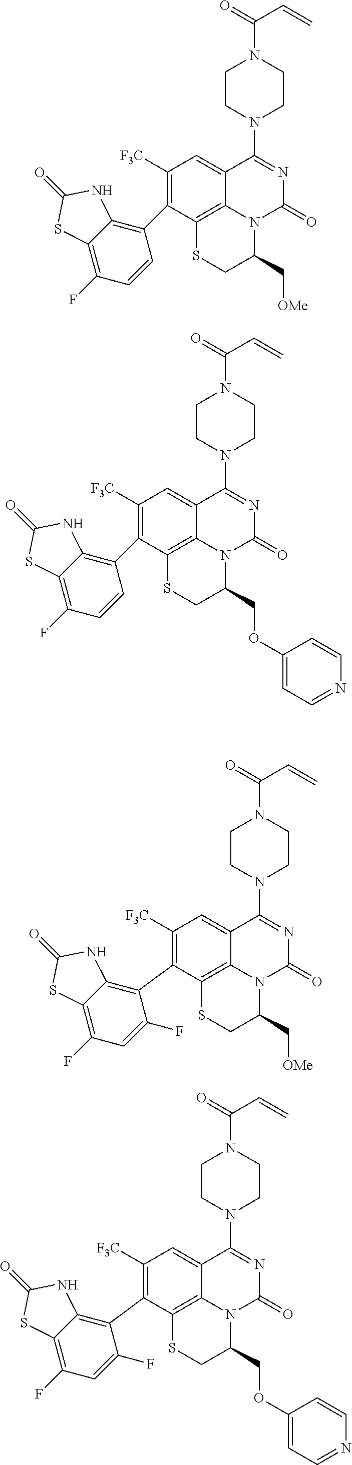 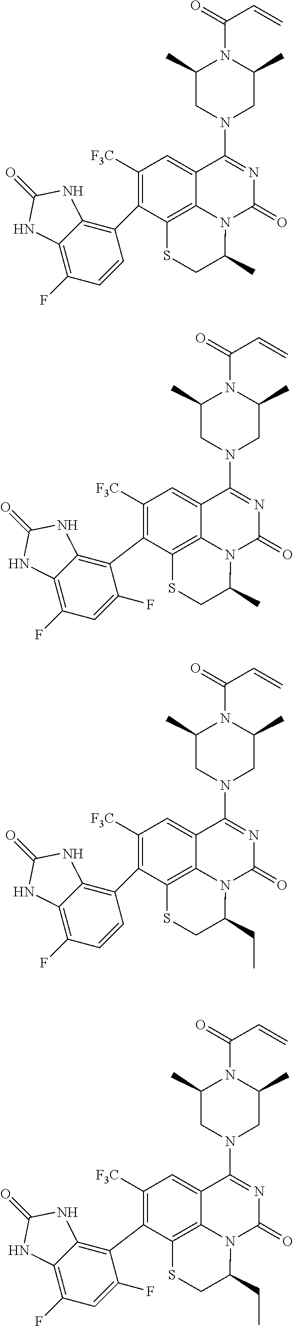 [SMILES: C=CC(=O)N1[C@H](C)CN(c2nc(=O)n3c4c(c(-c5ccc(F)c6[nH]c(=O)[nH]c56)c(C(F)(F)F)cc24)SC[C@@H]3C)C[C@@H]1C ; C=CC(=O)N1[C@H](C)CN(c2nc(=O)n3c4c(c(-c5c(F)cc(F)c6[nH]c(=O)[nH]c56)c(C(F)(F)F)cc24)SC[C@@H]3C)C[C@@H]1C ; C=CC(=O)N1[C@H](C)CN(c2nc(=O)n3c4c(c(-c5ccc(F)c6[nH]c(=O)[nH]c56)c(C(F)(F)F)cc24)SC[C@@H]3CC)C[C@@H]1C ; C=CC(=O)N1[C@H](C)CN(c2nc(=O)n3c4c(c(-c5c(F)cc(F)c6[nH]c(=O)[nH]c56)c(C(F)(F)F)cc24)SC[C@@H]3CC)C[C@@H]1C] 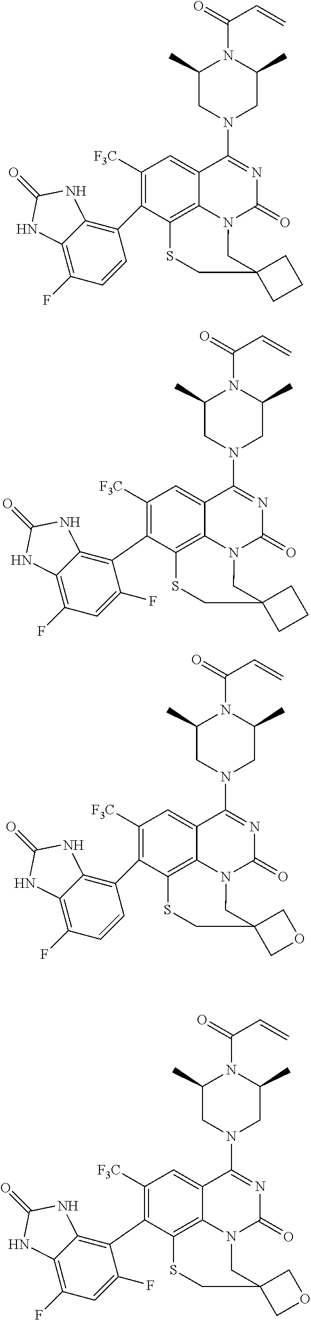 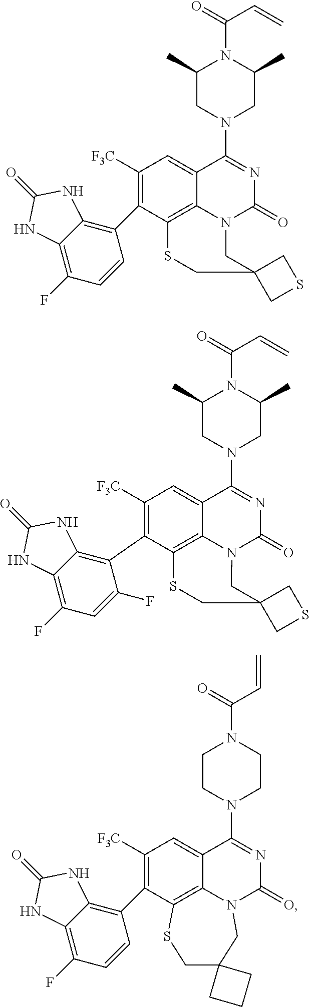 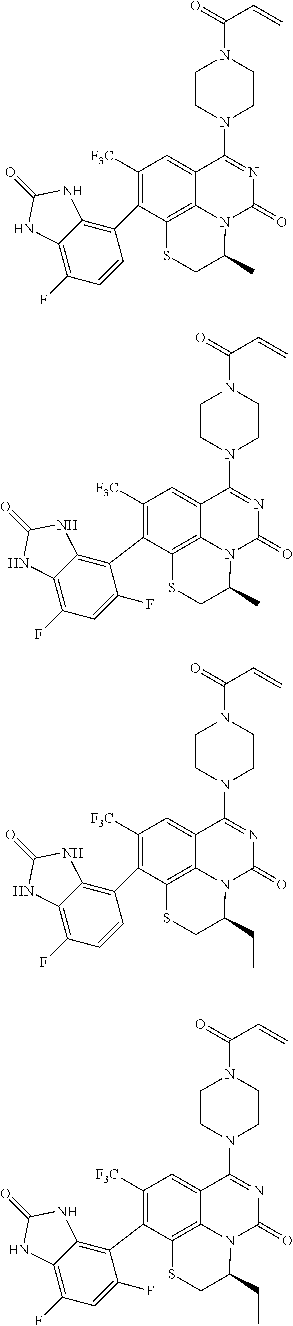 [SMILES: C=CC(=O)N1CCN(c2nc(=O)n3c4c(c(-c5ccc(F)c6[nH]c(=O)[nH]c56)c(C(F)(F)F)cc24)SC[C@@H]3C)CC1 ; C=CC(=O)N1CCN(c2nc(=O)n3c4c(c(-c5c(F)cc(F)c6[nH]c(=O)[nH]c56)c(C(F)(F)F)cc24)SC[C@@H]3C)CC1 ; C=CC(=O)N1CCN(c2nc(=O)n3c4c(c(-c5ccc(F)c6[nH]c(=O)[nH]c56)c(C(F)(F)F)cc24)SC[C@@H]3CC)CC1 ; C=CC(=O)N1CCN(c2nc(=O)n3c4c(c(-c5c(F)cc(F)c6[nH]c(=O)[nH]c56)c(C(F)(F)F)cc24)SC[C@@H]3CC)CC1] 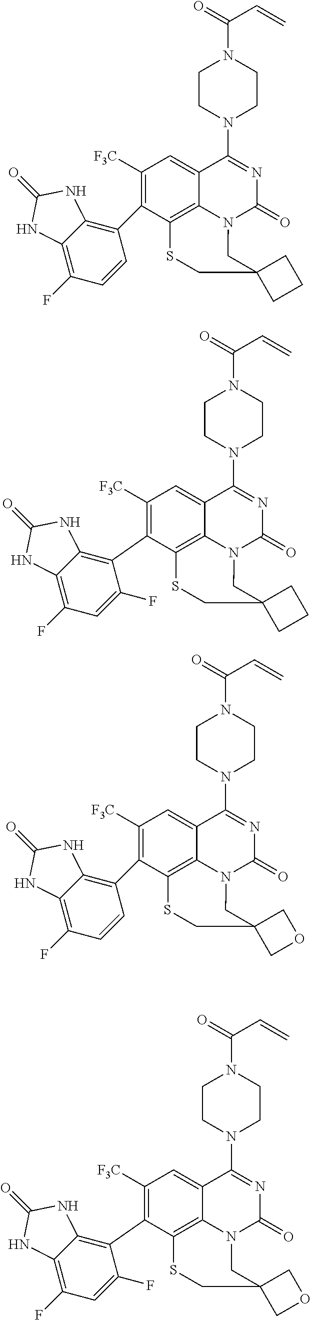 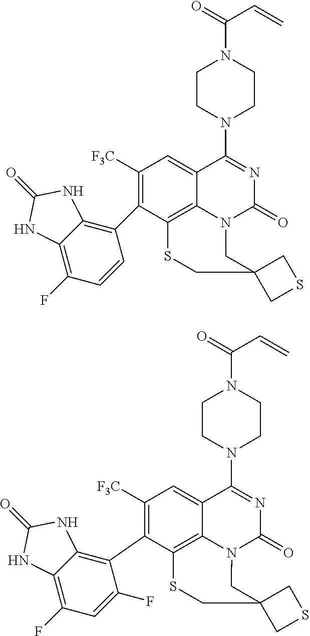 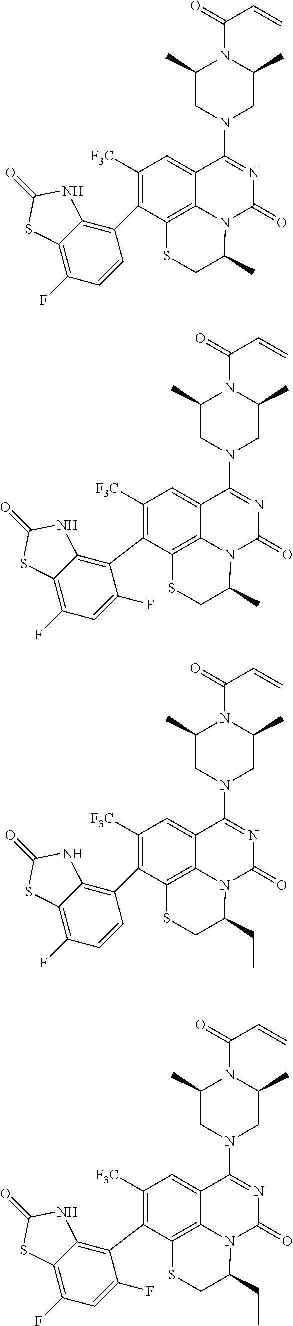 [SMILES: C=CC(=O)N1[C@H](C)CN(c2nc(=O)n3c4c(c(-c5ccc(F)c6sc(=O)[nH]c56)c(C(F)(F)F)cc24)SC[C@@H]3C)C[C@@H]1C ; C=CC(=O)N1[C@H](C)CN(c2nc(=O)n3c4c(c(-c5c(F)cc(F)c6sc(=O)[nH]c56)c(C(F)(F)F)cc24)SC[C@@H]3C)C[C@@H]1C ; C=CC(=O)N1[C@H](C)CN(c2nc(=O)n3c4c(c(-c5ccc(F)c6sc(=O)[nH]c56)c(C(F)(F)F)cc24)SC[C@@H]3CC)C[C@@H]1C ; C=CC(=O)N1[C@H](C)CN(c2nc(=O)n3c4c(c(-c5c(F)cc(F)c6sc(=O)[nH]c56)c(C(F)(F)F)cc24)SC[C@@H]3CC)C[C@@H]1C] 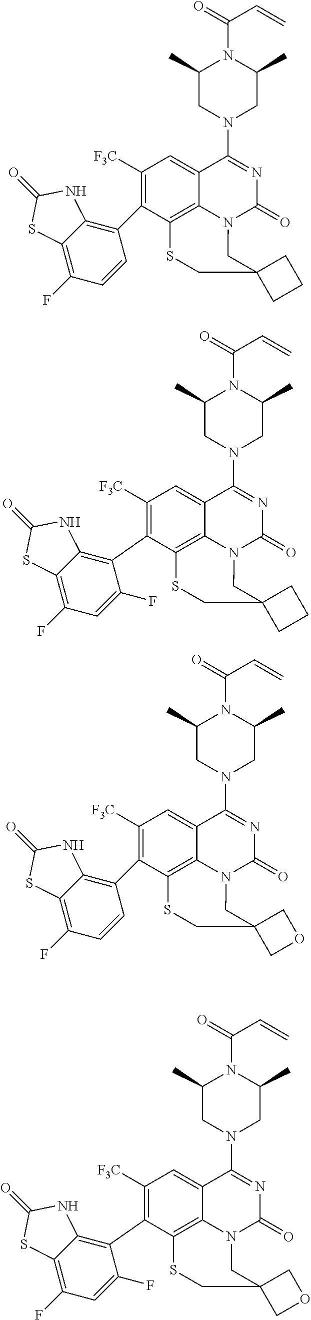 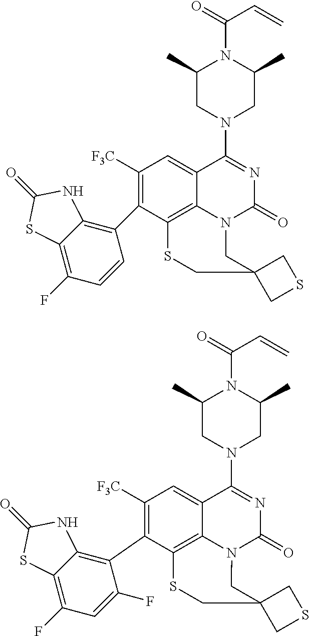 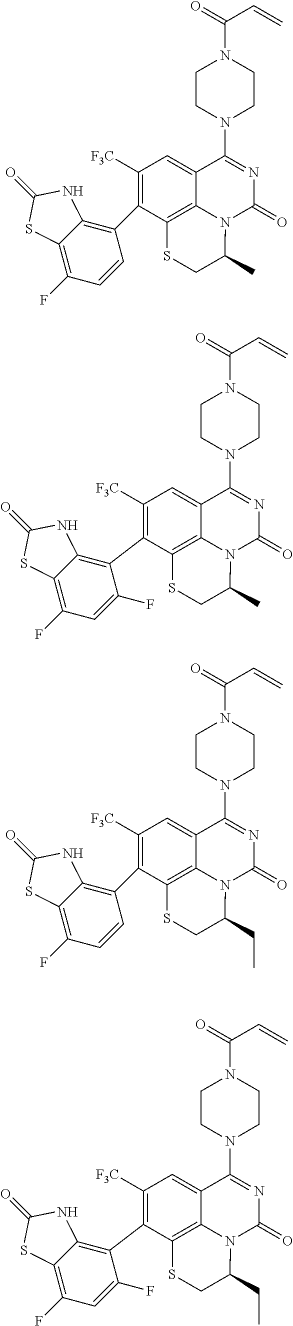 [SMILES: C=CC(=O)N1CCN(c2nc(=O)n3c4c(c(-c5ccc(F)c6sc(=O)[nH]c56)c(C(F)(F)F)cc24)SC[C@@H]3C)CC1 ; C=CC(=O)N1CCN(c2nc(=O)n3c4c(c(-c5c(F)cc(F)c6sc(=O)[nH]c56)c(C(F)(F)F)cc24)SC[C@@H]3C)CC1 ; C=CC(=O)N1CCN(c2nc(=O)n3c4c(c(-c5ccc(F)c6sc(=O)[nH]c56)c(C(F)(F)F)cc24)SC[C@@H]3CC)CC1 ; C=CC(=O)N1CCN(c2nc(=O)n3c4c(c(-c5c(F)cc(F)c6sc(=O)[nH]c56)c(C(F)(F)F)cc24)SC[C@@H]3CC)CC1] 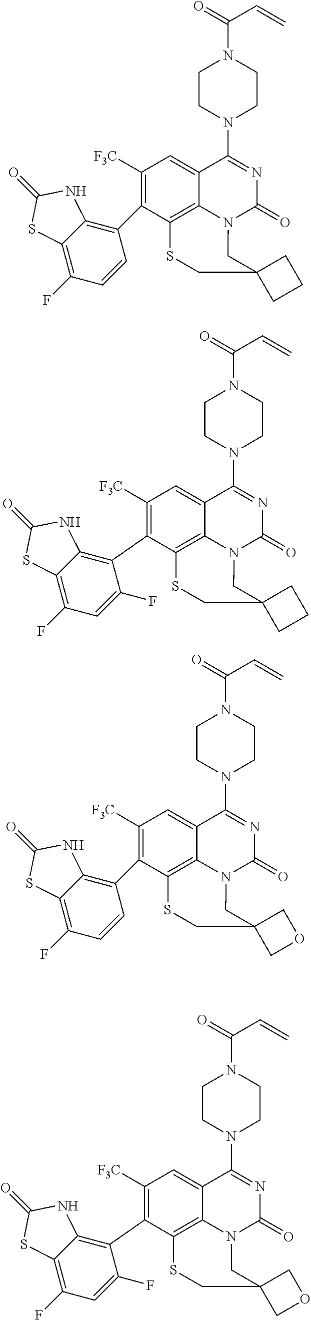 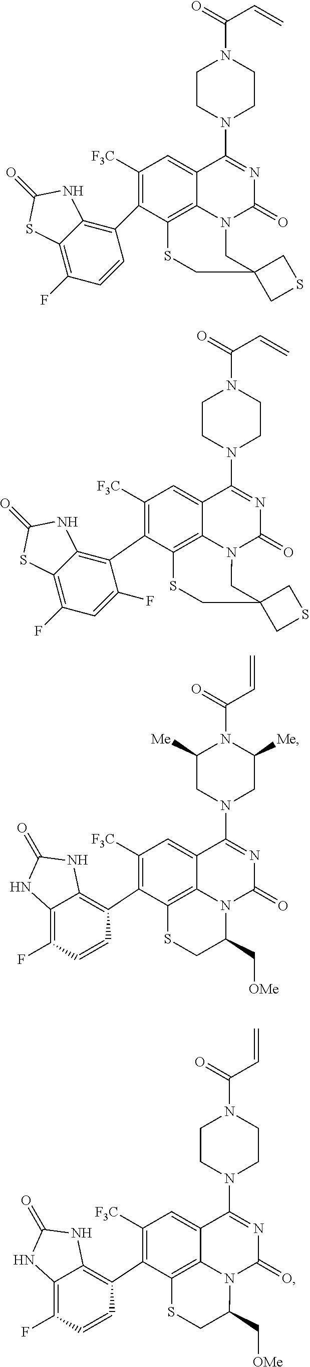 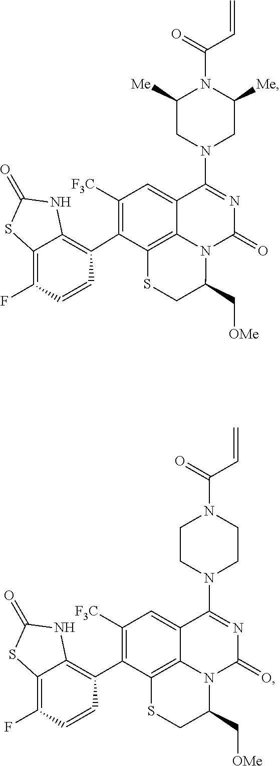
or pharmaceutically acceptable salt thereof.

16. A pharmaceutical composition comprising a pharmaceutically effective amount of a compound of any one of claims 1 to 15, or a pharmaceutically acceptable salt thereof, and a pharmaceutically acceptable excipient.

17. The pharmaceutical composition of claim 16, further comprising an additional therapeutic agent.

18. A method of treating a subject having cancer, the cancer characterized by the presence of a KRAS G12C mutation, the method comprising administering to the subject a therapeutically effective amount of a compound of any one of claims 1 to 15, or a pharmaceutically acceptable salt thereof, or a pharmaceutical composition thereof.

19. The method of claim 18, wherein the cancer is Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

20. The method of claim 18, wherein the cancer is non-small cell lung cancer, small cell lung cancer, colorectal cancer, rectal cancer, or pancreatic cancer.

21. Use of a compound of any one of claims 1 to 15, or a pharmaceutically acceptable salt thereof, or a a pharmaceutical composition thereof, in the manufacture of a medicament for the treatment of cancer in a subject, the cancer characterized by the presence of a KRAS G12C mutation.

22. The use of claim 21, wherein the cancer is Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

23. The use of claim 21, wherein the cancer is non-small cell lung cancer, small cell lung cancer, colorectal cancer, rectal cancer, or pancreatic cancer.


SEQUENCE LISTING (2 sequences)

SEQ ID NO: 1 - protein, 188 aa, Artificial Sequence
  Synthetic Polynulceotide
MAHHHHHHAGGAENLYFQSMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 170 aa, Artificial Sequence
  Synthetic Polynulceotide
SMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK